SEQUENCE LISTING (106 sequences)

SEQ ID NO: 1 - protein, 18 aa, Cottontail rabbit papillomavirus L2 protein
VGPLDIVPEVADPGGPTL

SEQ ID NO: 2 - protein, 17 aa, Cottontail rabbit papillomavirus L2 protein
PGGPTLVSLHELPAETP

SEQ ID NO: 3 - protein, 18 aa, Rabbit oral papillomavirus L2 protein
VGPLEVIPEAVDPAGSSI

SEQ ID NO: 4 - protein, 17 aa, Rabbit oral papillomavirus L2 protein
PAGSSIVPLEEYPAEIP

SEQ ID NO: 5 - protein, 13 aa, Human papillomavirus type 11 L2 protein
LIEESAIINAGAP

SEQ ID NO: 6 - protein, 13 aa, Human papillomavirus type 16 L2 protein
LVEETSFIDAGAP

SEQ ID NO: 7 - protein, 13 aa, Human papillomavirus type 18 L2 protein
LIEDSSVVTSGAP

SEQ ID NO: 8 - protein, 15 aa, Human papillomavirus type 16 L1 protein
GENVPDDLYIKGSGS

SEQ ID NO: 9 - protein, 20 aa, Human papillomavirus type 16 L1 protein
QPLGVGISGHPLLNKLDDTE

SEQ ID NO: 10 - protein, 9 aa, Ebola
VYKLDISEA

SEQ ID NO: 11 - protein, 11 aa, Ebola
DEQHHRRTDND

SEQ ID NO: 12 - protein, 9 aa, Ebola
LITNTIAGV

SEQ ID NO: 13 - protein, 7 aa, HIV Epitope
ELDKWAS

SEQ ID NO: 14 - protein, 16 aa, HIV Epitope
NEQELLELDKWASLWN

SEQ ID NO: 15 - protein, 14 aa, HIV Epitope
EQELLELDKWASLW

SEQ ID NO: 16 - protein, 6 aa, HIV Epitope
NWFDIT

SEQ ID NO: 17 - protein, 12 aa, HIV Epitope
LWNWFDITNWLW

SEQ ID NO: 18 - protein, 21 aa, HIV Epitope
LLELDKWASLWNWFDITNWLW

SEQ ID NO: 19 - protein, 9 aa, HIV Epitope
KSSGKLISL

SEQ ID NO: 20 - protein, 9 aa, HIV Epitope
CNGRLYCGP

SEQ ID NO: 21 - protein, 9 aa, HIV Epitope
GTKLVCFAA

SEQ ID NO: 22 - protein, 9 aa, HIV Epitope
CAGGLTCSV

SEQ ID NO: 23 - protein, 10 aa, HIV Epitope
SGRLYCHESW

SEQ ID NO: 24 - protein, 15 aa, HIV Epitope
HERSYMFSDLENRCI

SEQ ID NO: 25 - protein, 17 aa, HIV Epitope
TTSIRNKMQKEYALFYK

SEQ ID NO: 26 - protein, 18 aa, HIV Epitope
MEPVDPRLEPWKHPGSQP

SEQ ID NO: 27 - protein, 18 aa, HIV Epitope
MEPVDPKLEPWKHPGSQP

SEQ ID NO: 28 - protein, 18 aa, HIV Epitope
MEPVDPNLEPWKHPGSQP

SEQ ID NO: 29 - protein, 18 aa, HIV Epitope
MEPVDPNLEPWKHPGSQP

SEQ ID NO: 30 - protein, 18 aa, HIV Epitope
MDPVDPSLEPWKHPGSQP

SEQ ID NO: 31 - protein, 18 aa, HIV Epitope
MEPVDPSLEPWNHPGSQP

SEQ ID NO: 32 - protein, 14 aa, HIV Epitope
PTSQSRGDPTGPKE

SEQ ID NO: 33 - protein, 14 aa, HIV Epitope
PSSQPRGDPTGPKE

SEQ ID NO: 34 - protein, 14 aa, HIV Epitope
PASQSRGDPTGPTE

SEQ ID NO: 35 - protein, 14 aa, HIV Epitope
PLPRTQGDPTGSEE

SEQ ID NO: 36 - protein, 14 aa, HIV Epitope
PLPQTRGDPTGSKE

SEQ ID NO: 37 - protein, 14 aa, HIV Epitope
PLPTTRGNPTGPKE

SEQ ID NO: 38 - protein, 9 aa, HIV Epitope
LQARILAVE

SEQ ID NO: 39 - protein, 19 aa, HIV Epitope
LQARVLAVEGQARVLALER

SEQ ID NO: 40 - protein, 18 aa, HIV Epitope
EKNEQDLLALDKWASLWN

SEQ ID NO: 41 - protein, 14 aa, HIV Epitope
ADTIGPGRAFYTTK

SEQ ID NO: 42 - protein, 14 aa, HIV Epitope
ADTIGPGRAFYTTG

SEQ ID NO: 43 - protein, 14 aa, HIV Epitope
ADTIGPGQAFYAGG

SEQ ID NO: 44 - protein, 14 aa, Human Papillomavirus type 16 L2 protein
GLVEETSFIDAGAP

SEQ ID NO: 45 - protein, 14 aa, Human Papillomavirus type 18 L2 protein
GLIEDSSVVTSGAP

SEQ ID NO: 46 - protein, 7 aa, HIV Epitope (variation of SEQ ID NO: 16)
GNWFDIT

SEQ ID NO: 47 - protein, 14 aa, Human papillomavirus type 11 L2 protein (variation of SEQ ID NO: 5)
GLIEESAIINAGAP

SEQ ID NO: 48 - protein, 19 aa, Cottontail rabbit papillomavirus L2 protein (variation of SEQ ID NO: 1)
GVGPLDIVPEVADPGGPTL

SEQ ID NO: 49 - protein, 18 aa, Cottontail papillomavirus L2 protein
GPGGPTLVSLHELPAETP

SEQ ID NO: 50 - protein, 18 aa, Cottontail papillomavirus L2 protein
PGGPTLVSLHELPAETPY

SEQ ID NO: 51 - protein, 12 aa, Human Papilloma Virus
DRAHYNIVTFAG

SEQ ID NO: 52 - protein, 13 aa, Human Papilloma Virus
GDRAHYNIVTFAG

SEQ ID NO: 53 - protein, 14 aa, Human Papilloma Virus
AMDRAHYNIVTFAG

SEQ ID NO: 54 - protein, 14 aa, Human Papilloma Virus
QAEPDRAHYNIVTF

SEQ ID NO: 55 - protein, 15 aa, Human Papilloma Virus
GQAEPDRAHYNIVTF

SEQ ID NO: 56 - protein, 19 aa, Human Papilloma Virus
QAEPDRAHYNIVTFCCKCD

SEQ ID NO: 57 - protein, 20 aa, Human Papilloma Virus
GQAEPDRAHYNIVTFCCKCD

SEQ ID NO: 58 - protein, 8 aa, HIV
GELDKWAS

SEQ ID NO: 59 - protein, 15 aa, HIV
GEQELLELDKWASLW

SEQ ID NO: 60 - protein, 21 aa, HIV
GQPLGVGISGHPLLNKLDDTE

SEQ ID NO: 61 - protein, 16 aa, Human Papilloma Virus
GGENVPDDLYIKGSGS

SEQ ID NO: 62 - protein, 13 aa, HIV
MLQARILAVEAGA

SEQ ID NO: 63 - protein, 19 aa, HIV
GSPMLQARILAVEAGAGPS

SEQ ID NO: 64 - protein, 12 aa, HIV
LQARILAVEAGA

SEQ ID NO: 65 - protein, 22 aa, HIV
MLQARVLAVEGQARVLALEAGA

SEQ ID NO: 66 - protein, 28 aa, HIV
GSPMLQARVLAVEGQARVLALEAGAGPS

SEQ ID NO: 67 - protein, 21 aa, HIV
LQARVLAVEGQARVLALEAGA

SEQ ID NO: 68 - protein, 19 aa, Cottontail Rabbit Papilloma Virus
GVGPLEVIPEAVDPAGSSI

SEQ ID NO: 69 - protein, 18 aa, Cottontail Rabbit Papilloma Virus
GPAGSSIVPLEEYPAEIP

SEQ ID NO: 70 - protein, 18 aa, Cottontail Rabbit Papilloma Virus
PAGSSIVPLEEYPAEIPT

SEQ ID NO: 71 - protein, 17 aa, Rift Valley fever virus
MYKGTMDSGQTKREAGA

SEQ ID NO: 72 - protein, 23 aa, Rift Valley fever virus
GSPMYKGTMDSGQTKREAGAGPS

SEQ ID NO: 73 - protein, 16 aa, Rift Valley fever virus
YKGTMDSGQTKREAGA

SEQ ID NO: 74 - protein, 20 aa, HIV
MEPVDPRLEPWKHPGSQAGA

SEQ ID NO: 75 - protein, 26 aa, HIV
GSPMEPVDPRLEPWKHPGSQAGAGPS

SEQ ID NO: 76 - protein, 19 aa, HIV
EPVDPRLEPWKHPGSQAGA

SEQ ID NO: 77 - protein, 20 aa, HIV
MEPVDPSLEPWNHPGSQAGA

SEQ ID NO: 78 - protein, 26 aa, HIV
GSPMEPVDPSLEPWNHPGSQAGAGPS

SEQ ID NO: 79 - protein, 19 aa, HIV
EPVDPSLEPWNHPGSQAGA

SEQ ID NO: 80 - protein, 18 aa, HIV
MPTSQSRGDPTGPKEAGA

SEQ ID NO: 81 - protein, 24 aa, HIV
GSPMPTSQSRGDPTGPKEAGAGPS

SEQ ID NO: 82 - protein, 17 aa, HIV
PTSQSRGDPTGPKEAGA

SEQ ID NO: 83 - protein, 18 aa, HIV
MPLPTTRGNPTGPKEAGA

SEQ ID NO: 84 - protein, 24 aa, HIV
GSPMPLPTTRGNPTGPKEAGAGPS

SEQ ID NO: 85 - protein, 17 aa, HIV
PLPTTRGNPTGPKEAGA

SEQ ID NO: 86 - protein, 36 aa, HIV
GCTRPNYNKRKRIHIGPGRAFYTTKNIIGTIRQAHC

SEQ ID NO: 87 - protein, 15 aa, HIV
MIGPGRAFYTTGAGA

SEQ ID NO: 88 - protein, 21 aa, HIV
GSPMIGPGRAFYTTGAGAGPS

SEQ ID NO: 89 - protein, 14 aa, HIV
IGPGRAFYTTGAGA

SEQ ID NO: 90 - protein, 15 aa, HIV
MIGPGRAFYTTKAGA

SEQ ID NO: 91 - protein, 21 aa, HIV
GSPMIGPGRAFYTTKAGAGPS

SEQ ID NO: 92 - protein, 14 aa, HIV
IGPGRAFYTTKAGA

SEQ ID NO: 93 - protein, 15 aa, HIV
MIGPGQAFYAGGAGA

SEQ ID NO: 94 - protein, 21 aa, HIV
GDPMIGPGQAFYAGGAGAGPS

SEQ ID NO: 95 - protein, 14 aa, HIV
IGPGQAFYAGGAGA

SEQ ID NO: 96 - protein, 23 aa, Parvo Virus
MSDGAVQPDGGQPAVRNERATGS

SEQ ID NO: 97 - protein, 21 aa, Parvo Virus
MSDGAVQPDGGQPAVRNERAT

SEQ ID NO: 98 - protein, 20 aa, Parvo Virus
MSDGAVQPDGGQPAVRNERA

SEQ ID NO: 99 - protein, 16 aa, Parvo Virus
VQPDGGQPAVRNERAT

SEQ ID NO: 100 - protein, 13 aa, Rift Valley fever virus
KGTMDSGQTKREL

SEQ ID NO: 101 - protein, 17 aa, Ebola
HNTPVYKLDISEATQVE

SEQ ID NO: 102 - protein, 17 aa, Ebola
ATQVEQHHRRTDNDSTA

SEQ ID NO: 103 - protein, 17 aa, Ebola
GKLGLITNTIAGVAGLI

SEQ ID NO: 104 - protein, 6 aa, HIV
ELDKWA

SEQ ID NO: 105 - protein, 6 aa, Tobacco mosaic virus
CARYYA

SEQ ID NO: 106 - DNA, 23 bp, PCR Primer
ggagtttgtgtcggtgtgtattg

(19) United States
(12) Patent Application Publication
(10) Pub. No.: US 2004/0170606 A1
(43) Pub. Date: Sep. 2, 2004

(54) Production of peptides in plants as viral coat protein fusions

(76) Inventors: Kenneth E. Palmer, Vacaville, CA (US); Long V. Nguyen, Vacaville, CA (US); Rachel L. Toth, Fife (GB); Michael Jones, Dundee (GB); Sean Chapman, Fife (GB); Lisa Smolenska, Dundee (GB); Alison A. McCormick, Vacaville, CA (US); Gregory P. Pogue, Vacaville, CA (US)

Correspondence Address: WADDEY & PATTERSON, 414 UNION STREET, SUITE 2020, BANK OF AMERICA PLAZA, NASHVILLE, TN, 37219

(21) Appl. No.: 10/654,200

(22) Filed: Sep. 3, 2003

Related U.S. Application Data
(63) Continuation-in-part of application No. 10/457,082, filed on Jun. 6, 2003.
(60) Provisional application No. 60/386,921, filed on Jun. 7, 2002.
(60) Provisional application No. 60/407,795, filed on Sep. 3, 2002.

(51) Int. Cl. A61K 48/00; C12N 15/86

(52) U.S. Cl. 424/93.2; 435/456

(57) ABSTRACT

Vaccines and diagnostic composition are made and used for preventing, treating and detecting antigens from a papilloma virus, ebola virus, HIV virus, Rift Valley Fever virus or a parvovirus. The epitopes of these viruses are produced as genetically engineered fusion peptides in plants by infection with a recombinant tobamovirus vectors to express fusion proteins containing the epitope peptides.

### CROSS-REFERENCE TO RELATED APPLICATIONS

This application is a continuation-in-part of co-pending U.S. patent application Ser. No. 10/457,082 filed Jun. 6, 2003, entitled “FLEXIBLE VACCINE ASSEMBLY AND VACCINE DELIVERY PLATFORM”, which is incorporated herein by reference in its entirety, which claims the benefit of U.S. Provisional Application No. 60/386,921 filed Jun. 7, 2002, entitled “FLEXIBLE VACCINE ASSEMBLY AND VACCINE DELIVERY PLATFORM”, which is incorporated herein by reference in its entirety, and this application claims the benefit of U.S. Provisional Application No. 60/407,795, filed Sep. 3, 2002, entitled “DEVELOPMENT OF TILED PEPTIDE LIBRARY ON THE SURFACE OF A PLANT VIRUS AND EXPRESSION OF SPECIFIC EPITOPES OF HUMAN PAPILLOMAVIRUS AND HUMAN IMMUNODEFICIENCY VIRUS ON THE SURFACE OF A PLANT VIRUS”, which is incorporated herein by reference in its entirety.

### STATEMENT REGARDING FEDERALLY SPONSORED RESEARCH OR DEVELOPMENT

This invention was made with U.S. Government Support under cooperative agreement number 70NANB2H3048 awarded by the National Institute of Standards and Technology.

### BACKGROUND OF THE INVENTION

1. Field of the Invention

The present invention relates to the field of genetically engineered peptide production in plants, particularly to the use of tobamovirus vectors to express fusion proteins.

2. Description of Prior Art

Peptides are a diverse class of molecules having a variety of important chemical and biological properties. Some examples include; hormones, cytokines, immunoregulators, peptide-based enzyme inhibitors, vaccine antigens, adhesions, receptor binding domains, enzyme inhibitors and the like. The cost of chemical synthesis limits the potential applications of synthetic peptides for many useful purposes such as large scale therapeutic drug or vaccine synthesis. There is a need for inexpensive and rapid synthesis of milligram and larger quantities of naturally-occurring polypeptides. Towards this goal many animal and bacterial viruses have been successfully used as peptide carriers.

The safe and inexpensive culture of plants provides an improved alternative host for the cost-effective production of such peptides. During the last decade, considerable progress has been made in expressing foreign genes in plants. Foreign proteins are now routinely produced in many plant species for modification of the plant or for production of proteins for use after extraction. Animal proteins have been effectively produced in plants (reviewed in Krebbers et al., 1992).

Vectors for the genetic manipulation of plants have been derived from several naturally occurring plant viruses, including TMV (tobacco mosaic virus). TMV is the type member of the tobamovirus group. TMV has straight tubular virions of approximately 300 by 18 nm with a 4 nm-diameter hollow canal, consisting of approximately 2000 units of a single capsid protein wound helically around a single RNA molecule. Virion particles are 95% protein and 5% RNA by weight. The genome of TMV is composed of a single-stranded RNA of 6395 nucleotides containing five large ORFs. Expression of each gene is regulated independently. The virion RNA serves as the messenger RNA (mRNA) for the 5′ genes, encoding the 126 kDa replicase subunit and the overlapping 183 kDa replicase subunit that is produced by read through of an amber stop codon approximately 5% of the time. Expression of the internal genes is controlled by different promoters on the minus-sense RNA that direct synthesis of 3′-coterminal subgenomic mRNAs which are produced during replication (FIG. 1) Other tobamoviruses have a similar construction with genomic RNA of approximately 6.5 kb. The genomic RNA is used as an mRNA and translated to produce the replicase protein. These viruses may produce two replicase proteins, with the larger protein being produced by translational readthrough of an amber (AUG) stop codon. Both viruses produce two smaller coterminal subgenomic RNAs. The coat protein is encoded by the 3′-most RNA, and the movement proteins by the larger sgRNA. The virion RNA and sgRNAs are capped. Tobamovirus RNAs are not polyadenylated, but contain a tRNA-like structure at the 3′ end. Potevirus genomic and sgRNAs are polyadenylated. A detailed description of tobamovirus gene expression and life cycle can be found, among other places, in Dawson and Lehto, Advances in Virus Research 38:307-342 (1991).

For production of specific proteins, transient expression of foreign genes in plants using virus-based vectors has several advantages. Products of plant viruses are among the highest produced proteins in plants. Often a viral gene product is the major protein produced in plant cells during virus replication. Many viruses are able to quickly move from an initial infection site to almost all cells of the plant. Because of these reasons, plant viruses have been developed into efficient transient expression vectors for foreign genes in plants. Viruses of multicellular plants are relatively small, probably due to the size limitation in the pathways that allow viruses to move to adjacent cells in the systemic infection of entire plants. Most plant viruses have single-stranded RNA genomes of less than 10 kb. Genetically altered plant viruses provide one efficient means of transfecting plants with genes coding for peptide carrier fusions.

Human papillomaviruses (HPVs) are the etiologic agents of many benign and malignant tumors of stratified squamous epithelium (see recent reviews by Alani and Munger, 1998; zur Hausen, 1999; Einstein and Goldberg, 2002). In general, these tumors arise from keratinocytes of oral, epidermal, and anogenital sites, although some tumors (e.g. adenocarcinoma of the cervix) have a glandular morphology and origin. Not only do 95-99% of cervical cancers originate from papillomavirus-infected cells (zur Hausen 1999), but papillomaviruses also appear to contribute significantly to the development of oral and epidermal cancers (Balaram et al., 1995). Malignant conversion of cervical epithelium appears to be restricted to a “high risk” subset of papillomaviruses, whose association with cancer correlates with the ability of their E6 and E7 proteins to efficiently inactivate the cellular p53 and pRb tumor suppressor proteins, respectively. A single “high risk” HPV type, HPV-16 is associated with approximately 60% of cervical carcinomas. Papillomavirus infection has become a significant public health issue in the United States, where at least 17.9% of women are seropositive for HPV-16 infection (Stone et al., 2002); this figure does not include rates of infection with other “high risk” HPV types, and is still significantly lower than infection rates in developing countries. There is thus a great need for development of efficacious and cost-effective vaccines that will prevent papillomavirus infection and associated disease.

Papillomavirus are small (55 nm), non-enveloped, double-stranded DNA viruses with an 8 kb genome enclosed by a T=7 icosahedral capsid (Fields Virology text). Seven, or in some viruses eight early genes are involved in such processes as viral DNA replication (E1 and E2), RNA transcription (E2), and cell transformation (E5, E6, E7). The late genes encode the major capsid protein, L1, and the minor capsid protein, L2. The viral capsid is comprised of 72 pentamers, or capsomeres, of L1. Approximately 12 molecules of the L2 protein are associated with each capsid, probably at the capsid vertices. Regions of the L2 protein located towards the N-terminus are thought to be displayed on the surface of papillomavirus virions, since L2 antibodies can recognize both native virions and L1:L2 pseudovirions (Roden et al., 1994b; Liu et al., 1997; Kanawa et al., 1998a). The L2 protein interacts with the viral DNA and is probably involved in virion assembly (Day et al., 1998). Recombinant expression of the L1 protein in eukaryotic cells, e.g. in Sf9 insect cells using baculovirus expression vectors, results in the self-assembly of the L1 protein within the nuclear compartment into capsid-like structures termed “virus-like particles” or VLPs. Co-expression of L2 with L1 in eukaryotic expression systems results in incorporation of L2 into VLPs. Evidence suggests that L1:L2 VLPs are more stable than VLPs containing L1 alone (Kirnbauer et al., 1993). Papillomavirus L1:L2 VLPs can encapsidate plasmid DNA as well as genomic DNA from other papillomaviruses, and these pseudovirions have proven useful for development of surrogate infection assays that have allowed both antibody-mediated virus neutralization studies and investigation of the mechanism of papillomavirus binding and entry into host cells (Roden et al., 1996; Giroglou et al., 2001; Kawana et al., 1998b; 2001b). While L1 VLPs can efficiently bind the cell surface, pseudovirions containing L1 alone are much less efficient at DNA transfer than L1:L2 particles, implying that L2 plays a critical role in virus entry (Roden et al., 1997; Unckell et al., 1997).

Early efforts to express L1 protein-based vaccines showed that denatured protein purified from bacteria could not induce virus neutralizing antibodies in vaccinated animals. Conformational integrity of L1-based vaccines is critical because host antibodies recognized native, conformational epitopes on the virion (Ghim et al., 1991; Thompson et al., 1987). In the early to mid 1990's several groups demonstrated that L1 protein expressed in eukaryotic expression systems-recombinant baculovirus-transduced insect cells and yeast-could assemble into virus-like particles (VLPs) that retain conformational epitopes essential for induction of neutralizing antibodies. These purified VLPs were effective vaccines and protected rabbits, dogs and cattle from experimental infections (Suzich et al., 1995; Breitburd et al., 1995; Kirbauer et al., 1996). These results have been corroborated in several studies that show that sera from animals vaccinated with HPV L1 VLPs neutralize homologous HPV types in psuedovirus-based cell infection studies, and more recently that sera from participants in a HPV16 L1 VLP trial are also neutralizing (Schiller, 1999; Evans et al., 2001; Harro et al., 2001; Pastrana et al., 2001). Recent data show that small T=1 VLPs and L1 capsomere structures purified from bacteria expressing L1 fusion proteins retain many of the conformational epitopes that are required for effective L1 prophylactic vaccination, and this has been confirmed in the COPV model (Yuan et al. 2001).

Hemorrhagic fever viruses (HFVs) in the viral taxonomic families Filoviridae, Arenaviridae, Bunyaviridae and Flaviviridae threaten the health of humans and their livestock, particularly in developing countries. With the exception of yellow fever, there are no widely available, safe and efficacious vaccines that might prevent infection by any of the hemorrhagic fever viruses. In the wake of the attacks on the USA in September 2001, there is heightened awareness of the theoretical threat that biological terrorism, or biological warfare to human health. Given that HFVs were known to have been weaponized by the former Soviet Union, Russia, and the United States prior to 1969, development of safe, and easy-to-administer vaccines against high-priority HFVs would appear prudent from a National safety perspective (Borio et al., 2002). Certain of the HFVs, such as Rift Valley fever virus (RVFV) and Ebola virus (EBOV), present a threat to health of US military personnel deployed in Africa and the Middle East, as well as to travelers to those areas (Isaacson 2001).

Ideally, a vaccine designed to protect against infection with human immunodeficiency type 1 (HIV-1) will induce sterilizing immunity against a broad range of virus variants. However, generation of broadly-neutralizing antibodies (Nabs) by vaccination, let alone natural infection, has proven nearly impossible thus far. There have been some notable advances in development of vaccine regimens that are able to generate significant levels of protection against development of AIDS in non-human primate models (reviewed in 1,2,3,4). These vaccines allow animals to control viral challenge by strong priming of virus-specific CD8⁺ T-cells (cytotoxic T cells, CTLs). However, a CTL response alone cannot prevent infection, and mechanisms to induce Nabs that will neutralize a wide range of isolates remains a vital goal, especially in light of the fact that viral escape from vaccine-induced CTL control can sometimes occur (5). The Env spikes on the surface of the HIV-1 virion are the primary target for antibody-mediated neutralization. However, the Env proteins of HIV-1 are poorly antigenic, and generation of Nabs is difficult to achieve, probably because functionally important domains of the proteins are obscured by protein folding and carboydrate chains. Nevertheless, many infected people do mount a Nab response that is generally highly specific to the autologous virus, and not cross-neutralizing. This is not surprising given the phenomenal sequence and structural variation that is present in the Env proteins. However, a rare subset of infected individuals do produce broadly neutralizing Abs, which gives hope that induction of sterilizing immunity is possible.

The envelope proteins of T-cell line-adapted (TCLA) strains of HIV-1 elicit Nabs that mostly target linear epitopes in the third variable cysteine loop (V3 loop) of gp120, a region that is involved in co-receptor binding and hence vital for virus entry. Subtype C isolates of HIV-1, which infect more people worldwide than any other subtype, have relatively low level of sequence variation in the V3 loop (6,7). However, neutralization of subtype C virus by V3 loop Abs is not extremely efficient in vitro, perhaps reflecting poor immunogenicity of epitopes in this region (7). There is concern that the V3 loop may be hidden in the native gp120 structure and not accessible to the immune system, and therefore that generation of V3-specific Nabs will be difficult with gp120 subunit vaccines. However, the V3 loop is vital for viral entry, and so significant levels of V3 loop-targeted Nabs should help prevent transmission of HIV-1.

To date, six human monoclonal antibodies (Mabs) have been described that are capable of neutralizing a broad spectrum of HIV-1 variants in vitro. Three of these (IgGb12; 2G12 and 2F5) were described several years ago, and lend insight into the domains of the Env proteins that are important in viral entry, and thus for vaccine design. Monoclonal antibody “b12” recognizes a conformational epitope in the CD4 binding site of gp120; 2G12 recognizes a discontinuous epitope in the C2-V4 region of gp120 that includes N-glcyosylation sites, and 2F5 maps to a linear epitope (ELDKWA) in the membrane-proximal ectodomain of gp41 (9). Recently, two broadly neutralizing monoclonal antibodies 4E10 and Z13 were shown to recognize a continuous epitope with core sequence NWFDIT, just C-terminal to the 2F5 recognition sequence (10,11). This strongly indicates that the membrane proximal region of gp41 plays a critical role in virus entry. Another recently described monoclonal Fab was selected for binding to gp120-CD4-CCR5 complexes, and also displays a broad neutralization phenotype (12).

Passive transfer studies have shown that neutralizing Mabs are able to confer concentration-dependent sterilizing immunity to virus challenge by intravenous, oral and vaginal routes in Rhesus macaques. It is encouraging that the mAbs tested display significant synergy in their neutralization activity: this will reduce the minimum antibody concentration that is required for effective neutralization (reviewed in 13,14). A recent publication (15) demonstrates that MAb neutralizing activity can also be generated in vivo: in mice that expressed the gene for b12 from a recombinant adeno-associated virus vector. These studies on neutralizing Mabs have helped to demonstrate that one should be able to achieve significant levels of protection against HIV-1 infection and reduced rates of transmission of virus, if a way is found to induce robust production of Nabs in vaccinated animals and is incorporated into a vaccine regimen that includes strong priming of a CTL response.

In the light of the disappointing performance of whole Env-based vaccines, and the problems associated with poor immunogenicity of Env subunit vaccines, several studies have focused on the use of immunogens based on domains of Env proteins that are presumed targets for Abs. Data presented by Letvin et al. (8), that showed that antibodies induced against the V3 loop could provide partial protection against challenge with primary isolate-like SHIV-89.6 in Rhesus macaques. Efforts at generation of neutralizing antibodies with immunogens containing the core linear epitope recognized by the 2F5 antibody have been generally disappointing, with only non-neutralizing antibodies being produced (16,17). However, there is one notable exception: recently, Marusic et al. (18) showed that virus-like particles of the flexuous plant virus potato virus X (PVX) dispaying the 2F5 ELDKWA epitope could induce high levels of HIV-1 specific IgG and IgA in mice immunized with the recombinant virus-like particles (VLPs). This immunogen was able to induce production of human HIV-1 specific neutralizing antibodies (measured by in vitro inhibition of syncytium formation) in severe combined immunodeficient mice reconstituted with human periferal blood lymphocytes (hu-PBL-SCID) that had been immunized with human dendritic cells (DCs) pulsed with the PVX-2F5 VLPs. These authors speculate that presentation of the ELDKWAS sequence in a highly repetitive fashion on the surface of the PVX virion rendered the sequence highly immunogenic, and thus were able to generate Nabs. These results clearly warrant further investigation.

Until the recent discovery of the 4E10/Z3 human Mab, 2F5 was the only human Mab that appeared to recognize a linear epitope, and so peptides that could mimic the neutralizing epitope of b12 and 2G12 were not available for testing as potential immunogens. However, a linear peptide mimotope of the b12 epitope has recently been discovered using phage peptide display technology (19). This peptide (B2.1) appears to bind best to b12 when presented as a disulphide-linked homodimer on the surface of the phage. This phage particle is being optimized for use as an immunogen. Scala et al. (20) selected epitopes from libraries of peptides displayed on the surface of filamentous phage particles with sera from HIV⁺ patients, both from long term infected non-progressor donors and from donors who had progressed to AIDS illness. Five epitopes, presumed to be mimotopes of Env-specific neutralizing epitopes, were able to induce production of antibodies that neutralized TCLA HIV-1 strains IIIB and NL4-3, as well as the primary isolate AD8, but this less strongly than the TCLA strains (20). Subsequently, these authors showed that sera from individuals infected with all group M HIV-1 subgroups were able to recognize the phage-displayed mimotopes (21). Rhesus monkeys were immunized with phage particles displaying the five epitopes that had shown potentially protective immune responses in mice, and challenged with pathogenic SHIV-89.6PD. While the immunized animals were not protected from SHIV infection, there was evidence of significant control of the challenge virus and the monkeys were protected from progression to AIDS. These results show similar levels of control to vaccines designed to generate virus-specific CTLs and infer that the antibody response was able to control viremia in the challenged animals. A recent publication (22) described successful isolation of a number of human Nabs from XenoMouse immunized with gp120 derived from a primary Subtype B isolate (SF162). The authors noted potent neutralizing activity against the autologous virus isolate, and reactivity against both R5 and X4 isolates in Subtype B. The Nabs mapped to novel epitopes in domains known to possess neutralizing epitopes: V2-, V3- and CD4-binding domains of gp120, as well as in the C-terminal region of the V1 loop.

Some non-structural HIV-1 proteins, particularly Tat and Vpr, are found in the serum of infected individuals, and exert biological function, resulting in immunodeficiency and disease. The Tat protein is required for HIV-1 replication and pathogenesis. It is produced early in the viral life cycle. In the nucleus of the infected cell, it interacts with host factors and the TAR region of the viral RNA to enhance transcript elongation and to increase viral gene expression (Jeang et al., 1999). Tat also is also found extracellularly, where it has distinct functions that may indirectly promote virus replication and disease, either through receptor mediated signal transduction or after internalization and transport to the nucleus. Tat suppresses mitogen-, alloantigen- and antigen-induced lymphocyte proliferation in vitro by stimulating suppressive levels of alpha interferon and by inducing apoptosis in activated lymphocytes. In vivo, it is thought that Tat may alter immunity by upregulating IL-10 and reducing IL-12 production, or through its ability to increase chemokine receptor expression (Gallo et al., 2002; Tikhonov et al., 2003). Antibody production against Tat has, in some cases, correlated with delayed progression to AIDS in HIV-1 infected people (Gallo et al., 2002). Recently, Agwale et al. (2002) showed that antibodies induced in mice against a Tat protein subunit vaccine could negate the immune suppression activities of Tat in vivo. Subsequently, Tikhonov et al. (2003) identified linear epitopes on Tat that were reactive with Tat-neutralizing antibodies produced in vaccinated Rhesus macaques. From these data it is clear that antibodies that target the N-terminus, an internal basic domain, and the cell-binding domain of Tat (containing the integrin-binding motif “RGD”) can neutralize the extracellular version of Tat, and reduce the negative impact of Tat on the immune system.

Parvoviruses that are associated with enteric disease in domestic cats, dogs, mink and pigs are closely related antigenically, with different isolates diverging less than 2% in the sequence of the viral structural proteins. Vaccination with killed or live-attenuated parvovirus protects animals against infection by Feline panleukopenia virus (FPV), canine parvovirus (CPV), mink enteritis virus (MEV) and porcine parvovirus (PPV). However, maternal antibodies neutralize the vaccine, making it ineffective in animals that have not been weaned. Subunit vaccines might overcome this limitation, and provide useful alternatives to conventional vaccines.

### SUMMARY OF THE INVENTION

The present invention includes an immunological reagent having a plant viral protein covalently bound to an epitope peptide having the same linear sequence as an immunologically recognized epitope of a human papilloma virus, human immunodeficiency virus, ebola virus, rift valley fever virus or parvovirus.

The present invention also includes an immunological reagent having a plant viral protein covalently bound to an epitope peptide having the same linear sequence as an immunologically recognized epitope of a human papilloma virus, human immunodeficiency virus, ebola virus, rift valley fever virus or parvovirus, wherein the epitope peptide contains a sequence selected from the group consisting of the peptide sequences of Table 1, the peptide sequences of Table 6, the peptide sequences of Table 7, the peptide sequences of Table 8,

HNTPVYKLDISEATQVE, ATQVEQHHRRTDNDSTA, GKLGLITNTIAGVAGLI,
VQPDGGQPAVRNERAT, MSDGAVQPDGGQPAVRNERA,
MSDGAVQPDGGQPAVRNERAT and KGTMDSGQTKREL.

The invention also includes a vaccine having an immunological reagent having a plant viral protein covalently bound to an epitope peptide having the same linear sequence as an immunologically recognized epitope of a human papilloma virus, human immunodeficiency virus, ebola virus, rift valley fever virus or parvovirus, wherein the epitope peptide contains a sequence selected from the group consisting of the peptide sequences of Table 1, the peptide sequences of Table 6, the peptide sequences of Table 7, the peptide sequences of Table 8, HNTPVYKLDISEATQVE, ATQVEQHHRRTDNDSTA, GKLGLITNTIAGVAGLI, VQPDGGQPAVRNERAT, MSDGAVQPDGGQPAVRNERA, MSDGAVQPDGGQPAVRNERAT and KGTMDSGQTKREL, and a pharmaceutically acceptable carrier or excipient.

The present invention also includes a method for eliciting an immune response in an animal by administering a vaccine having an immunological reagent having a plant viral protein covalently bound to an epitope peptide having the same linear sequence as an immunologically recognized epitope of a human papilloma virus, human immunodeficiency virus, ebola virus, rift valley fever virus or parvovirus, wherein the epitope peptide contains a sequence selected from the group consisting of the peptide sequences of Table 1, the peptide sequences of Table 6, the peptide sequences of Table 7, the peptide sequences of Table 8, HNTPVYKLDISEATQVE, ATQVEQHHRRTDNDSTA, GKLGLITNTIAGVAGLI, VQPDGGQPAVRNERAT, MSDGAVQPDGGQPAVRNERA, MSDGAVQPDGGQPAVRNERAT and KGTMDSGQTKREL, and a pharmaceutically acceptable carrier or excipient to the animal.

The present invention includes a virus-like particle having a plurality of assembled protein subunits wherein each protein subunit is a plant viral coat protein covalently bound to an epitope peptide having the same linear sequence as an immunologically recognized epitope of a human papilloma virus, human immunodeficiency virus, ebola virus, rift valley fever virus or parvovirus.

The present invention also includes a virus-like particle having a plurality of assembled protein subunits wherein each protein subunit is a plant viral coat protein covalently bound to an epitope peptide having the same linear sequence as an immunologically recognized epitope of a human papilloma virus, human immunodeficiency virus, ebola virus, rift valley fever virus or parvovirus, wherein the sequence selected from the group consisting of the peptide sequences of Table 1, the peptide sequences of Table 6, the peptide sequences of Table 7, the peptide sequences of Table 8, HNTPVYKLDISEATQVE, ATQVEQHHRRTDNDSTA, GKLGLITNTIAGVAGLI, VQPDGGQPAVRNERAT, MSDGAVQPDGGQPAVRNERA, MSDGAVQPDGGQPAVRNERAT and KGTMDSGQTKREL.

The invention includes a vaccine having a virus-like particle having a plurality of assembled protein subunits wherein each protein subunit is a plant viral coat protein covalently bound to an epitope peptide having the same linear sequence as an immunologically recognized epitope of a human papilloma virus, human immunodeficiency virus, ebola virus, rift valley fever virus or parvovirus, and a pharmaceutically acceptable carrier or excipient.

The invention also includes a method for eliciting an immune response in an animal including administering the vaccine having a virus-like particle having a plurality of assembled protein subunits wherein each protein subunit is a plant viral coat protein covalently bound to an epitope peptide having the same linear sequence as an immunologically recognized epitope of a human papilloma virus, human immunodeficiency virus, ebola virus, rift valley fever virus or parvovirus, and a pharmaceutically acceptable carrier or excipient to the animal.

The invention includes a plant virus having at least one plant viral coat protein covalently bound to an epitope peptide having the same linear sequence as an immunologically recognized epitope of a human papilloma virus, human immunodeficiency virus, ebola virus, rift valley fever virus or parvovirus.

The invention also includes a plant virus having at least one plant viral coat protein covalently bound to an epitope peptide having the same linear sequence as an immunologically recognized epitope of a human papilloma virus, human immunodeficiency virus, ebola virus, rift valley fever virus or parvovirus, wherein the sequence sequence is selected from the group consisting of the peptide sequences of Table 1, the peptide sequences of Table 6, the peptide sequences of Table 7, the peptide sequences of Table 8, HNTPVYKLDISEATQVE, ATQVEQHHRRTDNDSTA, GKLGLITNTIAGVAGLI, VQPDGGQPAVRNERAT, MSDGAVQPDGGQPAVRNERA, MSDGAVQPDGGQPAVRNERAT and KGTMDSGQTKREL.

The present invention also includes a vaccine having a plant virus having at least one plant viral coat protein covalently bound to an epitope peptide having the same linear sequence as an immunologically recognized epitope of a human papilloma virus, human immunodeficiency virus, ebola virus, rift valley fever virus or parvovirus, wherein the sequence sequence is selected from the group consisting of the peptide sequences of Table 1, the peptide sequences of Table 6, the peptide sequences of Table 7, the peptide sequences of Table 8, HNTPVYKLDISEATQVE, ATQVEQHHRRTDNDSTA, GKLGLITNTIAGVAGLI, VQPDGGQPAVRNERAT, MSDGAVQPDGGQPAVRNERA, MSDGAVQPDGGQPAVRNERAT and KGTMDSGQTKREL and a pharmaceutically acceptable carrier or excipient.

The invention also includes a method for eliciting an immune response in an animal including administering a vaccine having a plant virus having at least one plant viral coat protein covalently bound to an epitope peptide having the same linear sequence as an immunologically recognized epitope of a human papilloma virus, human immunodeficiency virus, ebola virus, rift valley fever virus or parvovirus, wherein the sequence sequence is selected from the group consisting of the peptide sequences of Table 1, the peptide sequences of Table 6, the peptide sequences of Table 7, the peptide sequences of Table 8, HNTPVYKLDISEATQVE, ATQVEQHHRRTDNDSTA, GKLGLITNTIAGVAGLI, VQPDGGQPAVRNERAT, MSDGAVQPDGGQPAVRNERA, MSDGAVQPDGGQPAVRNERAT and KGTMDSGQTKREL and a pharmaceutically acceptable carrier or excipient to the animal.

The present invention also includes the composition of the sixth paragraph of this section or the composition of the tenth paragraph of this section containing a plurality of different epitope peptides, each on a separate plant viral coat protein molecule.

The present invention also includes a method for preparing an antibody against a papilloma virus, ebola virus, HIV virus, Rift Valley Fever virus or a parvovirus including: exposing an animal to the vaccine described in the third, seventh, or eleventh paragraph of this section, recovering cells or body fluids from the animal, and preparing an antibody from said cells or body fluids.

The present invention includes the method of the above paragraph wherein the antibody is neutralizing.

The present invention includes a method for detecting a papilloma virus, ebola virus, HIV virus, Rift Valley Fever virus or a parvovirus comprising contacting an antibody produced by the method of the 14^{th} paragraph of this section with a sample suspecting of containing a virus, and detecting the presence or absence of antibody binding to the virus.

The present invention includes a method for inducing an immune response in an animal against a peptide epitope including: coupling the peptide epitope to a first carrier antigen to make a first vaccine composition, coupling the peptide epitope to a second carrier antigen, which is different from the first carrier antigen, to make a second vaccine composition, immunizing the animal with the first vaccine composition, at a later time, immunizing the animal with the second vaccine composition, wherein the immune response to the peptide epitope is boosted greater than the boosting of either carrier antigen.

The present invention also includes the method according to the previous paragraph further including: coupling a second peptide epitope to a third carrier antigen to make a third vaccine composition, coupling the second peptide epitope to a fourth carrier antigen, which is different from the third carrier antigen but may be the same as either the first carrier antigen or the second carrier antigen, to make a fourth vaccine composition, immunizing an individual animal with the first vaccine composition and the third composition, at a later time, immunizing the same individual animal with the second vaccine composition and the fourth composition, wherein the immune responses to the first and second peptide epitope are boosted greater than the boosting of the carrier antigens.

It is still another object of the present invention to provide polynucleotides encoding the genomes of the subject recombinant plant viruses.

It is another further object of the present invention to provide the coat fusion proteins encoded by the subject recombinant plant viruses.

It is yet another further object of the present invention to provide plant cells that have been infected by the recombinant plant viruses of the invention.

### BRIEF DESCRIPTION OF THE DRAWINGS

FIG. 1. Tobamovirus gene map and expression products are diagrammed.

FIG. 2. A series of flow charts showing methods used for construction of recombinant tobamoviruses with useful peptides genetically fused to the coat protein gene

FIG. 3. An uninfected Glurk plant leaf is shown on the left and a leaf with lesions is shown on the right, where each necrotic local lesion indicates a virus infection event.

FIG. 4: SDS PAGE and MALDI-TOF analysis. The vaccine samples were run in triplicate, with the Mark12 protein molecular weight markers (Invitrogen) in the fourth lane in every case. The molecular weight marker bands, from top to bottom are 36.5 kDa; 31 kDa; 21.5 kDa and 14.4 kDa. The molecular weight of the upper viral band, as determined by MALDI-TOF is indicated in the figure.

FIG. 5: Western blot analysis of TMV:papillomavirus vaccines. Samples were loaded as indicated in the coomassie blue stained gel (lower right) and probed with rabbit antisera indicated above the blots.

FIG. 6: Scatter plot indicating ELISA (IgG) response of all immunized animals to the cognate peptide antigen. Sera analyzed here were from bleed 3, post vaccine 4.

FIG. 7: Bar graph showing responses to peptide antigens, pooled data with error bars indicating 95% confidence interval. Sera analyzed were from bleed 3, post vaccine 4.

FIG. 8: Analysis of serum cross-reactivity between papillomavirus peptide antigens.

FIG. 9: Comparison of IgG antibody response to vaccination with CRPV2.1 vaccines, BEI treated and non-treated (left) and to the HPV6/11 vaccine (right). Each bar represents the specific IgG level of an individual mouse.

FIG. 10: shows the results of IgG subtype measurement in sera of animals vaccinated with the five different papillomavirus L2 vaccines. The immune response appears balanced; but, the concentration of IgG1 subtype appears to be at least 3-fold greater than that of IgG2, perhaps indicating a dominant Th2 response.

FIG. 11: ELISA measurement of relative amounts peptide specific IgG after vaccine 3 (left) and 4 (right)

FIG. 12: IgG subtype measurements in sera of Guinea Pigs vaccinated with TMV:papillomavirus vaccines.

FIG. 13: Cross-reactivity of sera of guinea pigs immunized with CRPV- or HPV 6/11 TMV peptide fusions, against HPV 16 L2 peptide capture antigen (LVEETSFIDAGAP). Each bar indicates the antibody response induced in an individual animal. The dashed line indicates the probable level of non-specific cross-reactive antibodies that were induced on vaccination with TMV virions carrying the very distantly related cottontail rabbit papillomavirus peptide 2.1. FIG. 14, below, illustrates the amino acid identity between these three peptides.

FIG. 14: Shared amino acid identity between the HPV-11 L2 peptide present on recombinant TMV virion LSB2282; the CRPV 2.1 peptide present on recombinant TMV virion LSB2283, and the HPV-16 L2 peptide LVEETSFIDAGAP that was conjugated to bovine serum albumin and used as the capture antigen in the ELISA.

FIG. 15: Solubility of example coat fusion proteins carrying Ebola epitopes. Photograph of SDS-PAGE gel of crude proteins extracts from plants inoculated with infectious transcripts carrying the Ebola epitope-coat protein fusions.

### DESCRIPTION OF THE PREFERRED EMBODIMENTS

An “immunologically recognized epitope peptide” generally has at least 8 amino acids unique to an antigen, or closely related antigens, and is a binding site for a specific antibody or T-cell receptor. The antibody and/or cytotoxic T-lymphocyte containing the T-cell receptor are induced upon immunization or infection with an antigen containing this epitope peptide.

An “epitope peptide” or a “peptide epitope” includes the specific sequences described below chemically bonded to the N-terminal, the C-terminal or an internal region of an antigen. The epitope peptide may be longer than the specific sequences described below with boardering sequence(s) having the same sequence as the viral pathogen's antigens. The epitope may contain slight amino acid substitutions (preferably conservative substitutions) slight deletions in the sequences recited provided that the epitope peptide contains a sufficient amount of the sequence to bind to a specific antibody and/or to elicit a specific antibody capable of binding specifically to the natural antigen. Examples of a shorter epitope peptide include the 1 N-terminal amino acid in the HPV-16 L1 protein epitope and Ebola virus epitope GP-1 amino acid number 405.

The term “protein” is intended to also encompass derivitized molecules such as glycoproteins and lipoproteins as well as lower molecular weight polypeptides.

The terms “binding component”, “ligand” or “receptor” may be any of a large number of different molecules, and the terms are sometimes usable interchangeably. In the context of the present invention the receptor is usually an antibody and the ligand is usually the pathogenic virus such as a papilloma virus, ebola virus, HIV virus, Rift Valley Fever virus or a parvovirus.

The term “bind” includes any physical attachment or close association, which may be permanent or temporary. Generally, an interaction of hydrogen bonding, hydrophobic forces, van der Waals forces etc. facilitates physical attachment between the ligand molecule of interest and the receptor. The “binding” interaction may be brief as in the situation where binding causes a chemical reaction to occur. Reactions resulting from contact between the binding component and the analyte are within the definition of binding for the purposes of the present invention. Binding is preferably specific. Specific binding indicates substantially no strong binding to other antigens. A comparison of the binding of different papilloma viruses as shown below emphasizes the nature of the specific binding. The binding may be reversible, particularly under different conditions.

The term “bound to” refers to a tight coupling of the two components mentioned. The nature of the binding may be chemical coupling through a linker moiety, as a fusion protein produced by expression of a single ORF, physical binding or packaging such as in a macromolecular complex. Likewise, all of the components of a cell are “bound to” the cell.

“Labels” include a large number of directly or indirectly detectable substances bound to another compound and are known per se in the immunoassay and hybridization assay fields. Examples include radioactive, fluorescent, enzyme, chemiluminescent, hapten, a solid phase, spin labels, particles, etc. Labels include indirect labels, which are detectable in the presence of another added reagent, such as a receptor bound to a biotin label and added avidin or streptavidin, labeled or subsequently labeled with labeled biotin simultaneously or later.

An “antibody” is a typical receptor and includes fragments of antibodies, e,g, Fab, Fab2, recombinant, reassortant, single chain, phage display and other antibody variations. The receptor may be directly or indirectly labeled.

In situations where a chemical label is not used in an assay, alternative methods may be used such as agglutination or precipitation of the ligand/receptor complex, detecting molecular weight changes between complexed and uncomplexed ligands and receptors, optical changes to a surface and other changes in properties between bound and unbound ligands or receptors.

The term “biological sample” includes tissues, fluids, solids (preferably suspendable), extracts and fractions that contain proteins. These protein samples are from cellular or fluids originating from an organism. In the present invention, the host is generally a mammal, most preferably a human.

The present invention provides recombinant plant viruses that express fusion proteins that are formed by fusions between a plan viral coat protein and protein of interest. By infecting plant cells with the recombinant plant viruses of the invention, relatively large quantities of the protein of interest may be produced in the form of a fusion protein. The fusion protein encoded by the recombinant plant virus may have any of a variety of forms. The protein of interest may be fused to the amino terminus of the viral coat protein or the protein of interest may be fused to the carboxyl terminus of the viral coat protein. In other embodiments of the invention, the protein of interest may be fused internally to a coat protein. The viral coat fusion protein may have one or more properties of the protein of interest. The recombinant coat fusion protein may be used as an antigen for antibody development or to induce a protective immune response.

The subject invention provides novel recombinant plant viruses that code for the expression of fusion proteins that consist of a fusion between a plant viral coat protein and a protein of interest. The recombinant plant viruses of the invention provide for systemic expression of the fusion protein, by systemically infecting cells in a plant. Thus by employing the recombinant plant viruses of the invention, large quantities of a protein of interest may be produced.

The fusion proteins of the invention comprise two portions: (i) a plant viral coat protein and (ii) a protein of interest. The plant viral coat protein portion may be derived from the same plant viral coat protein that serves a coat protein for the virus from which the genome of the expression vector is primarily derived, i.e., the coat protein is native with respect to the recombinant viral genome. Alternatively, the coat protein portion of the fusion protein may be heterologous, i.e., non-native, with respect to the recombinant viral genome. In a preferred embodiment of the invention, the 17.5 KDa coat protein of tobacco mosaic virus is used in conjunction with a tobacco mosaic virus derived vector. The protein of interest portion of the fusion protein for expression may consist of a peptide of virtually any amino acid sequence, provided that the protein of interest does not significantly interfere with (1) the ability to bind to a receptor molecule, including antibodies and T cell receptors (2) the ability to bind to the active site of an enzyme (3) the ability to induce an immune response, (4) hormonal activity, (5) immunoregulatory activity, and (6) metal chelating activity. The protein of interest portion of the subject fusion proteins may also possess additional chemical or biological properties that have not been enumerated. Protein of interest portions of the subject fusion proteins having the desired properties may be obtained by employing all or part of the amino acid residue sequence of a protein known to have the desired properties. For example, the amino acid sequence of hepatitis B surface antigen may be used as a protein of interest portion of a fusion protein invention so as to produce a fusion protein that has antigenic properties similar to hepatitis B surface antigen. Detailed structural and functional information about many proteins of interest are well known; this information may be used by the person of ordinary skill in the art so as to provide for coat fusion proteins having the desired properties of the protein of interest. The protein of interest portion of the subject fusion proteins may vary in size from one amino acid residue to over several hundred amino acid residues, preferably the sequence of interest portion of the subject fusion protein is less than 100 amino acid residues in size, more preferably, the sequence of interest portion is less than 50 amino acid residues in length. It will be appreciated by those of ordinary skill in the art that, in some embodiments of the invention, the protein of interest portion may need to be longer than 100 amino acid residues in order to maintain the desired properties. Likewise, it will be appreciated that a smaller sequence containing only the particular epitope or even a fraction of it may be used. Preferably, the size of the protein of interest portion of the fusion proteins of the invention is minimized (but retains the desired biological/chemical properties), when possible.

While the protein of interest portion of fusion proteins of the invention may be derived from any of the variety of proteins, proteins for use as antigens are particularly preferred. For example, the fusion protein, or a portion thereof, may be injected into a mammal, along with suitable adjutants, so as to produce an immune response directed against the protein of interest portion of the fusion protein. The immune response against the protein of interest portion of the fusion protein has numerous uses, such uses include, protection against infection, and the generation of antibodies useful in immunoassays.

The location (or locations) in the fusion protein of the invention where the viral coat protein portion is joined to the protein of interest is referred to herein as the fusion joint. A given fusion protein may have one or two fusion joints. The fusion joint may be located at the carboxyl terminus of the coat protein portion of the fusion protein (joined at the amino terminus of the protein of interest portion). The fusion joint may be located at the amino terminus of the coat protein portion of the fusion protein (joined to the carboxyl terminus of the protein of interest). In other embodiments of the invention, the fusion protein may have two fusion joints. In those fusion proteins having two fusion joints, the protein of interest is located internal with respect to the carboxyl and amino terminal amino acid residues of the coat protein portion of the fusion protein, i.e., an internal fusion protein. Internal fusion proteins may comprise an entire plant virus coat protein amino acid residue sequence (or a portion thereof) that is “interrupted” by a protein of interest, i.e., the amino terminal segment of the coat protein portion is joined at a fusion joint to the amino terminal amino acid residue of the protein of interest and the carboxyl terminal segment of the coat protein is joined at a fusion joint to the amino terminal acid residue of the protein of interest.

When the coat fusion protein for expression is an internal fusion protein, the fusion joints may be located at a variety of sites within a coat protein. Suitable sites for the fusion joints may be determined either through routine systematic variation of the fusion joint locations so as to obtain an internal fusion protein with the desired properties. Suitable sites for the fusion jointly may also be determined by analysis of the three dimensional structure of the coat protein so as to determine sites for “insertion” of the protein of interest that do not significantly interfere with the structural and biological functions of the coat protein portion of the fusion protein. Detailed three dimensional structures of plant viral coat proteins and their orientation in the virus have been determined and are publicly available to a person of ordinary skill in the art. For example, a resolution model of the coat protein of Cucumber Green Mottle Mosaic Virus (a coat protein bearing strong structural similarities to other tobamovirus coat proteins) and the virus can be found in Wang and Stubbs J. Mol. Biol. 239:371-384 (1994). Detailed structural information on the virus and coat protein of Tobacco Mosaic Virus can be found, among other places in Namba et al, J. Mol. Biol. 208:307-325 (1989) and Pattanayek and Stubbs J. Mol. Biol. 228:516-528 (1992).

Knowledge of the three dimensional structure of a plant virus particle and the assembly process of the virus particle permits the person of ordinary skill in the art to design various coat protein fusions of the invention, including insertions, and partial substitutions. For example, if the protein of interest is of a hydrophilic nature, it may be appropriate to fuse the peptide to the TMVCP (Tobacco mosaic tobamovirus coat protein) region known to be oriented as a surface loop region. Likewise, alpha helical segments that maintain subunit contacts might be substituted for appropriate regions of the TMVCP helices or nucleic acid binding domains expressed in the region of the TMVCP oriented towards the genome.

Polynucleotide sequences encoding the subject fusion proteins may comprise a “leaky” stop codon at a fusion joint. The stop codon may be present as the codon immediately adjacent to the fusion joint, or may be located close (e.g., within 9 bases) to the fusion joint. A leaky stop codon may be included in polynucleotides encoding the subject coat fusion proteins so as to maintain a desired ratio of fusion protein to wild type coat protein. A “leaky” stop codon does not always result in translational termination and is periodically translated. The frequency of initiation or termination at a given start/stop codon is context dependent. The ribosome scans from the 5′-end of a messenger RNA for the first ATG codon. If it is in a non-optimal sequence context, the ribosome will pass, some fraction of the time, to the next available start codon and initiate translation downstream of the first. Similarly, the first termination codon encountered during translation will not function 100% of the time if it is in a particular sequence context. Consequently, many naturally occurring proteins are known to exist as a population having heterogeneous N and/or C terminal extensions. Thus by including a leaky stop codon at a fusion joint coding region in a recombinant viral vector encoding a coat fusion protein, the vector may be used to produce both a fusion protein and a second smaller protein, e.g., the viral coat protein. A leaky stop codon may be used at, or proximal to, the fusion joints of fusion proteins in which the protein of interest portion is joined to the carboxyl terminus of the coat protein region, whereby a single recombinant viral vector may produce both coat fusion proteins and coat proteins. Additionally, a leaky start codon may be used at or proximal to the fusion joints of fusion proteins in which the protein of interest portion is joined to the amino terminus of the coat protein region, whereby a similar result is achieved. In the case of TMVCP, extensions at the N and C terminus are at the surface of viral particles and can be expected to project away from the helical axis. An example of a leaky stop sequence occurs at the junction of the 126/183 kDa reading frames of TMV and was described over 15 years ago (Pelham, H. R. B., 1978). Skuzeski et al. (1991) defined necessary 3′ context requirements of this region to confer leakiness of termination on a heterologous protein marker gene (beta-glucuronidase) as CAR-YYA (C=cytidine, A=adenine, Y=pyrimidine).

In another embodiment of the invention, the fusion joints on the subject coat fusion proteins are designed so as to comprise an amino acid sequence that is a substrate for protease. By providing a coat fusion protein having such a fusion joint, the protein of interest may be conveniently derived from the coat protein fusion by using a suitable proteolytic enzyme. The proteolytic enzyme may contact the fusion protein either in vitro or in vivo.

The expression of the subject coat fusion proteins may be driven by any of a variety of promoters functional in the genome of the recombinant plant viral vector. In a preferred embodiment of the invention, the subject fusion proteins are expressed from plant viral subgenomic promoters using vectors as described in U.S. Pat. No. 5,316,931.

Recombinant DNA technologies have allowed the life cycle of numerous plant RNA viruses to be extended artificially through a DNA phase that facilitates manipulation of the viral genome. These techniques may be applied by the person ordinary skill in the art in order make and use recombinant plant viruses of the invention. The entire cDNA of the TMV genome was cloned and functionally joined to a bacterial promoter in an E. coli plasmid (Dawson et al., 1986). Infectious recombinant plant viral RNA transcripts may also be produced using other well known techniques, for example, with the commercially available RNA polymerases from T7, T3 or SP6. Precise replicas of the virion RNA can be produced in vitro with RNA polymerase and dinucleotide cap, m7GpppG. This not only allows manipulation of the viral genome for reverse genetics, but it also allows manipulation of the virus into a vector to express foreign genes. A method of producing plant RNA virus vectors based on manipulating RNA fragments with RNA ligase has proved to be impractical and is not widely used (Pelcher, L. E., 1982). Detailed information on how to make and use recombinant RNA plant viruses can be found, among other places in U.S. Pat. No. 5,316,931 (Donson et al.), which is herein incorporated by reference. The invention provides for polynucleotide encoding recombinant RNA plant vectors for the expression of the subject fusion proteins. The invention also provides for polynucleotides comprising a portion or portions of the subject vectors. The vectors described in U.S. Pat. No. 5,316,931 are particularly preferred for expressing the fusion proteins of the invention.

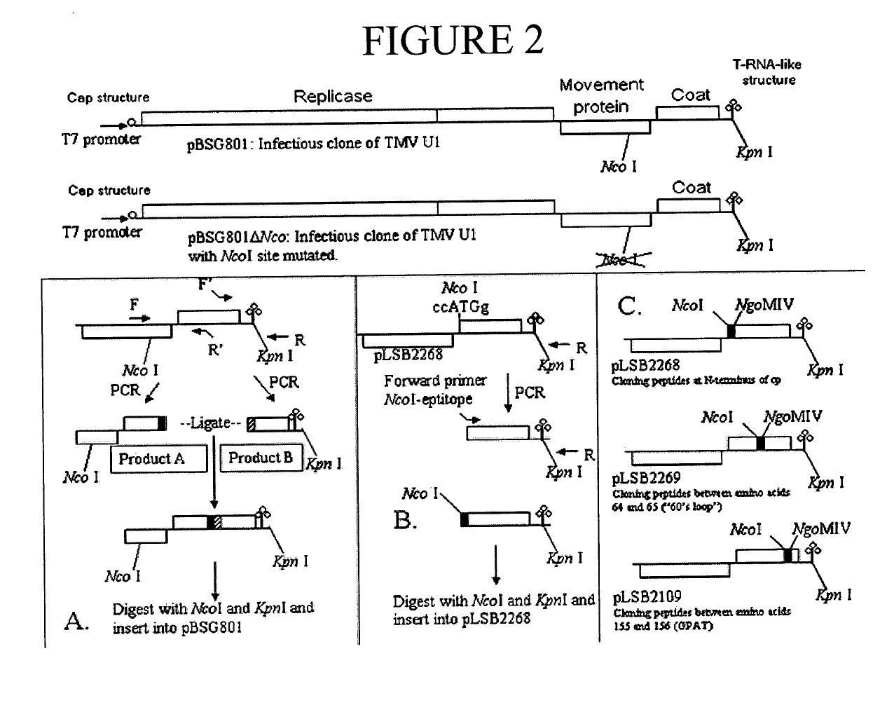

FIG. 2 demonstrates one way used in the present invention for constructing the recombinant tobamoviruses used in the present invention. An infectious clone of TMV strain U1 called pBSG801 was used as the basic vector for construction of peptide fusion constructs, as well as for building other peptide fusion-acceptor vectors. In some cases, an NcoI restriction site was required for peptide insertions. A version of pBSG801 was created where the NcoI site in the movement protein gene was mutated, without altering the amino acid sequence of the movement protein. In this construct (pBSG801ΔNco), NcoI is available as a cloning site. A. shows a method that was used for construction of peptide fusion constructs using a PCR-ligation method. PCR primers F (GGAGTTTGTGTCGGTGTGTATTG)and R (GGAGTTTGTGTCGGTGTGTATTG) amplify a fragment of the pBSG801 or plasmid that spans the 3′ end of the viral genome to a point upstream of the native NcoI site within the movement protein open reading frame. Peptides may be fused to internal positions in the coat protein open reading frame by addition of synthetic DNA encoding the a fragment of the peptide of interest to internal primers F′ and R′. Primers F and R′ and R and F′ are then used to amplify PCR products A and B. Ligation of A and B reconstitutes the peptide of interest in the same reading frame as the coat protein. The ligated product is digested with NcoI and KpnI The engineered coat protein-peptide fusion is then translated in vivo when in vitro-generated infectious RNA is used to infect Nicotiana plants. B. Shows part of the plasmid pLSB2268 which was generated from pBSG801ΔNco: an NcoI site (CCATGG) was inserted at the start of the coat protein open reading frame to facilitate cloning of N-terminal peptide fusions by PCR. Synthetic DNA encoding peptides of interest was inserted in frame with the ATG in the NcoI site into a primer homologous with the 5′1 end of the coat protein gene. The specific PCR primer was used in PCR reactions with primer R (GGAGTTTGTGTCGGTGTGTATTG) and resulting PCR product was digested with NcoI and KpnI and cloned into pLSB2268. An alternative strategy for insertion of synthetic DNA encoding peptides of interest in different positions of tobamovirus coat proteins is shown in C. Three different vectors were created; all were derived from pBSG801ΔNco. These acceptor vectors, pLSB2268; pLSB2269 and pLSB2109 contain restriction sites suitable for accepting double stranded oligonucleotides with sticky ends compatible with NcoI (5′) and NgoMIV (3′). Complementary single stranded oligonucleotides are synthesized that encode the peptide of interest, such that the sense (top) strand has the sequence 5′-CATG(NNN)ₙG-3′ and the antisense (bottom) strand has the sequence 5′-CCGGC(NNN)ₙ-3′ where (NNN)ₙ denotes a sequence of DNA that encodes amino acids in the peptide of interest. The complementary oligonucleotides are annealed in vitro and the resulting dsDNA oligonucleotide with overhanging CATG and CCGG ends is ligated with acceptor vector that has been digested with NcoI and NgoMIV to create various coat protein fusion constructs.

In addition to providing the described viral coat fusion proteins, the invention also provides for virus particles that comprise the subject fusion proteins. The coat of the virus particles of the invention may consist entirely of coat fusion protein. In another embodiment of the virus particles of the invention, the virus particle coat may consist of a mixture of coat fusion proteins and non-fusion coat protein, wherein the ratio of the two proteins may be varied. As tobamovirus coat proteins may self-assemble into virus particles, the virus particles of the invention may be assembled either in vivo or in vitro. The virus particles may also be conveniently dissassembled using well known techniques so as to simplify the purification of the subject fusion proteins, or portions thereof.

The invention also provides for recombinant plant cells comprising the subject coat fusion proteins and/or virus particles comprising the subject coat fusion proteins. These plant cells may be produced either by infecting plant cells (either in culture or in whole plants) with infectious virus particles of the invention or with polynucleotides encoding the genomes of the infectious virus particle of the invention. The recombinant plant cells of the invention have many uses. Such uses include serving as a source for the fusion coat proteins of the invention.

The protein of interest portion of the subject fusion proteins may comprise many different amino acid residue sequences, and accordingly may have different possible biological/chemical properties however, in a preferred embodiment of the invention the protein of interest portion of the fusion protein is useful as a vaccine antigen. The surface of TMV particles and other tobamoviruses contain continuous epitopes of high antigenicity and segmental mobility thereby making TMV particles especially useful in producing a desired immune response. These properties make the virus particles of the invention especially useful as carriers in the presentation of foreign epitopes to mammalian immune systems.

While the recombinant RNA viruses of the invention may be used to produce numerous coat fusion proteins for use as vaccine antigens or vaccine antigen precursors, it is of particular interest to provide vaccines against viral pathogens of humans, and domestic animals. It is of particular interest to provide vaccines against human papillomavirus (HPV) types that are implicated in the etiology of cervical cancer, and other neoplasias, including but not limited to HPV-16, HPV-18, HPV-31, HPV-33, HPV-35 and HPV-52. While not implicated in cervical cancer a vaccine against HPV-6 and HPV-11 is also desirable as such viruses cause much disease. It is also of particular interest to provide vaccines against hemorrhagic fever-causing viruses such as Rift Valley fever virus (RVFV) and Ebola viruse (EBOV), as these pathogens present significant threat to the US population if weaponized by terrorists. In addition, it is of interest to provide vaccines against human immunodeficiency virus type 1 (HIV-1), and against parvoviruses that are significant pathogens of human companion animals (particularly cats and dogs), and livestock (especially pigs).

When the fusion proteins of the invention, portions thereof, or viral particles comprising the fusion proteins are used in vivo, the proteins are typically administered in a composition comprising a pharmaceutical carrier. A pharmaceutical carrier can be any compatible, non-toxic substance suitable for delivery of the desired compounds to the body. Sterile water, alcohol, fats, waxes and inert solids may be included in the carrier. Pharmaceutically accepted adjuvants (buffering agents, dispersing agent) may also be incorporated into the pharmaceutical composition. Additionally, when the subject fusion proteins, or portion thereof, are to be used for the generation of an immune response, protective or otherwise, formulation for administration may comprise one or immunological adjuvants in order to stimulate a desired immune response.

When the fusion proteins of the invention, or portions thereof, are used in vivo, they may be administered to a subject, human or animal, in a variety of ways. The pharmaceutical compositions may be administered orally or parenterally, i.e., subcutaneously, intramuscularly or intravenously. Thus, this invention provides compositions for parenteral administration which comprise a solution of the fusion protein (or derivative thereof) or a cocktail thereof dissolved in an acceptable carrier, preferably an aqueous carrier. A variety of aqueous carriers can be used, e.g., water, buffered water, 0.4% saline, 0.3% glycerine and the like. These solutions are sterile and generally free of particulate matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example sodium acetate, sodium chloride, potassium chloride, calcium chloride, sodium lactate, etc. The concentration of fusion protein (or portion thereof) in these formulations can vary widely depending on the specific amino acid sequence of the subject proteins and the desired biological activity, e.g., from less than about 0.5%, usually at or at least about 1% to as much as 15 or 20% by weight and will be selected primarily based on fluid volumes, viscosities, etc., in accordance with the particular mode of administration selected.

Actual methods for preparing parenterally administrable compositions and adjustments necessary for administration to subjects will be known or apparent to those skilled in the art and are described in more detail in, for example, Remington's Pharmaceutical Science, current edition, Mack Publishing Company, Easton, Pa., which is incorporated herein by reference.

The invention having been described above, may be better understood by reference to the following examples. The examples are offered by way of illustration and are not intended to be interpreted as limitations on the scope of the invention.

The vaccine compositions of the present invention are used for inducing an immune response to prevent infection by one or more of the pathogenic viruses. When the infection is of a long duration such as with HPV and HIV, the vaccines may be provided to help in clearing the infection or to suppress the infection. Generally, vaccines are given by injection or contact with mucosal, buccal, lung, eye or similar tissues. Transdermal and oral administration may be used when sufficiently adsorbed and stable, particularly when tolerization is desired.

One or more of the vaccines may be used cross-immunize the individual recipient against related strains or viruses. Likewise, a single vaccine designed against one pathogen may be used against other related ones. For example, a single parvovirus vaccine composition may be used to induce an immune response against feline, canine and porcine parvoviruses in cats, dogs and pigs respectively due to a very similar viral antigen common to each virus. The peptide epitope containing compositions may also be used as positive controls for diagnostic, epidemiological and other screening purposes.

The same compositions as used for vaccines may be used to immunize an animal for the production of antibodies, antibody-secreting cells (e.g. for monoclonal antibody production), T-cell receptors and corresponding T-cells. These materials may be used for diagnostic purposes, given by injection to provide passive immunity prophalactically or to treat an active infection.

A number of different binding assay formats may be used to detect the pathogenic viruses or antibodies to the viruses as a measure of past infection. Both competitive and non-competitive assays may be used with direct or indirect labels to one or more binding partners. These binding assays, particularly immunoassays are well known in the art.

### EXAMPLE 1

### Papillomavirus Vaccines

Antigens are most effectively delivered to the immune system in a repetitive configuration, like that presented by virus-like particles. For B cell responses, a crucial factor for immunogenicity is repetitiveness and order of antigenic determinants. Many viruses display a quasicrystalline surface with a regular array of epitopes which efficiently crosslink antigen-specific immunoglobulins on the surface of B cells, leading to B cell proliferation and production of secreted antibodies (Bachmann et al., 1993; Fehr et al., 1998). Triggered B cells can activate helper T cells, leading to long-lived B cell memory—essential for any vaccine. In part due to these observations, and because only very low levels of L2-specific antibodies are detected in vaccinated or infected animals, only L1 VLP vaccines have been pursued in clinical trials of prophylactic vaccines. However, because VLP and capsomeric L1 vaccines induce mainly type-specific neutralizing antibodies, a comprehensive solution to HPV prophylactic vaccination probably requires vaccination with L1 from multiple types.

The dominant virus neutralizing immune response against HPV-16 particles is directed against a conformational epitope, described by the monoclonal antibody named V5 (Christensen et al.,1996). There are, in addition, two linear epitopes in HPV-16 L1 that may induce antibodies capable of neutralization of other papillomavirus types; these two epitopes (QPLGVGISGHPLLNKLDDTE and ENVPDDLYIKGSGS) bind monoclonal antibodies I23 and J4, respectively. Unfortunately, the immune response that is generated to L1-derived VLP vaccines is a dominant type-specific neutralizing response. If there were ways to enhance the recognition of the sub-dominant epitopes that might induce antibodies with a broader specificity against other papillomavirus types, this method could be incorporated into a vaccine regimen to generate a protective immune response against multiple high risk papillomavirus types. The cross-neutralizing epitopes 1-23 and J-4 were displayed on the surface of TMV particles as shown in Table 1. Other peptide fusion vaccines are also shown in Table 1.

TABLE 1
TMV - Papillomavirus Peptide Fusion Vaccines

| Construct | | | |
|---|---|---|---|
| Name | Virus Name | Origin of Peptide | Peptide Sequence |
| LSB2283 (GPAT) | TMV:CRPV2.1 | Cottontail rabbit | VGPLDIVPEVADPGGPTL |
| | | papillomavirus | |
| | | L2 protein | |
| LSB2288 (GPAT) | TMV:CRPV2.2 | Cottontail rabbit | PGGPTLVSLHELPAETP |
| | | papillomavirus | |
| | | L2 protein | |
| LSB2285 (GPAT) | TMV:ROPV2.1 | Rabbit oral | VGPLEVIPEAVDPAGSSI |
| | | papillomavirus | |
| | | L2 protein | |
| LSB2280 (GPAT) | TMV:ROPV2.2 | Rabbit oral | PAGSSIVPLEEYPAEIP |
| | | papillomavirus | |
| | | L2 protein | |
| LSB2282 (GPAT) | TMV:HPV-11 L2 | Human papilloma- | LIEESAIINAGAP |
| | | virus type | |
| | | 11 L2 protein | |
| LSB2406 (N-ter) | | | |
| LSB2278 (GPAT) | TMV:HPV-16 L2 | Human papilloma- | LVEETSFIDAGAP |
| | | virus type | |
| | | 16 L2 protein | |
| LSB2291 (N-ter) | | | |
| LSB2281 (GPAT) | TMV:HPV-18 L2 | Human papilloma- | LIEDSSVVTSGAP |
| | | virus type | |
| | | 18 L2 protein | |
| LSB2297 (N-ter) | | | |
| LSB2284 (GPAT) | TMV:HPV-16J4 | Human papilloma- | GENVPDDLYIKGSGS |
| | | virus type | |
| | | 16 L1 protein | |
| LSB2404 (N-ter) | | | |
| LSB2279 (GPAT) | TMV:HPV-16I23 | Human papilloma- | QPLGVGISGHPLLNKLDDTE |
| | | virus type | |
| | | 16 L1 protein | |
| | TMV wild type | N/A | N/A |

Antibodies against the N-terminus of L2 can be neutralizing in pseudoinfection studies, but paradoxically the neutralizing antibodies do not inhibit virion binding to the cell surface (Gaukroger et al., 1996; Roden et al., 1994). It is possible that domains of L2 that bind neutralizing antibodies are not accessible in native virions or pseudovirions, but are exposed at some point during viral entry into cells. Recently Kawana et al. (2001b) showed that amino acids 108-126 of HPV 16 L2 (a neutralizing domain) could bind a proteinaceous receptor, present at higher level on the surface of epithelial cells than non-epithelial cells. These data suggest that L2 binds a co-receptor on the cell surface and that at least a subset of virus neutralizing antibodies can block L2-mediated virus entry. Papillomavirus virions and pseudovirions bind a wide variety of cell types and the N-termini of L2 proteins of mucosotropic papillomaviruses show high homology. In the light of these facts it is tempting to speculate that the binding specificity between L2 and a papillomavirus cell surface coreceptor could be a determinant of papillomavirus tissue tropism.

The data of Kawana and colleagues show that immunization of mice (Kawana et al., 1999; 2001) and humans (Kawana et al., 2003) with the 13 amino acid HPV-16 L2-derived peptide (sequence: LVEETSFIDAGAP) could induce antibodies that can neutralize papillomavirus infection in vitro. Importantly, sera from animals and humans immunized with this peptide can neutralize the homologous virus (HPV-16) as well as related mucosotropic viruses: HPV-11; HPV-6 and HPV52 (Kawana et al., 1999; 2001; 2003). These results are very significant, since this is the first time that antibodies from animals immunized with papillomavirus antigens have shown cross-type neutralization activity. Kawana et al. (2003) had to deliver relatively large quantities of peptide—500 μg, by the intranasal route, to induce papillomavirus L2-specific antibodies. The inventors predicted that display of the peptide as a highly repetitive antigen array, such as on the surface of TMV, would enhance the immunogenicity of the peptide.

Genetic Fusion of Papillomavirus Peptides to the Coat Protein of Tobacco mosaic Virus Strain U1

Tobacco mosaic virus strain U1 (vulgare) was used as the carrier for peptide fusions. All peptides were fused near the carboxy-terminus of the U1 coat protein, at a position four residues before the carboxy terminal amino acids (GPAT). DNA sequences encoding the papillomavirus epitopes were synthesized in PCR primers and a PCR strategy was used to fuse the sequences to the TMV coat protein at a position four amino acids from the C-terminus (position “GPAT”) or at the N-terminus, immediately after the initiating methionine (“N-ter”). A synthetic DNA sequence encoding the L2 peptide of interest was inserted into the U1 coat protein DNA sequence, by PCR with specific primers and fragment ligation. Recombinant TMV clones were sequenced, and clones with DNA sequences that matched predicted sequences were assigned clone identifiers, as indicated in Table 1.

Infection of Plants with Infectious Chimeric TMV:Papillomavirus Clones

The plasmids described in Table 1 were transcribed in vitro to generate capped infectious RNA transcripts (mMESSAGE mMACHINE Kit, Ambion, Austin Tex.). Transcription reactions were diluted in FES buffer, and plants were inoculated by leaf abrasion. The four rabbit papillomavirus constructs (pLSB2283, pLSB2288, pLSB2285 and pLSB2280) were inoculated on two leaves of each of 40 to 46 Nicotiana benthamiana plants, 24 days post-sowing, and infectious transcripts of pLSB2282 (TMV:HPV-11L2) were inoculated on two leaves of each of 40, 27 day-old, Nicotiana excelsiana plants, a Large Scale Biology Corporation-proprietary field host for TMV (Fitzmaurice W P, U.S. Pat. No. 6,344,597). Wild type TMV U1 was prepared from infected tobacco (Nicotiana tabaccum). The recombinant TMV:ROPV2.2 virus induced necrotic symptoms on infected N. benthamiana plants; the other recombinant viruses induced symptoms typically seen in Nicotiana plants infected with TMV coat protein fusions, i.e. leaf crinkling, bubbling and twisting, and a stunted plant growth habit. The number of grams of tissue and DPI for each construct is summarized in Table 2.

TABLE 2
Record of production of recombinant TMV in Nicotiana plants

| | | | # | Tissue |
|---|---|---|---|---|
| Virus Name | Plant Species | DPI | plants | weight |
| TMV:CRPV2.1 | Nicotiana | 8 | 60 | 247 g |
| | Benthamiana | | | |
| TMV:HPV-11 | Excelsiana | 11 | 45 | 267 g |
| L2 | | | | |
| TMV:ROPV2.2* | Nicotiana | 10 | 90 | 143 g |
| | Benthamiana | | | |
| TMV wild Type** | MD609 | 15 | 12 | 258 g |
| TMV:CRPV2.2 | Nicotiana | 11 | 81 | 269 g |
| | Benthamiana | | | |
| TMV:ROPV2.1 | Nicotiana | 10 | 81 | 281 g |
| | Benthamiana | | | |

*very severe viral symptoms- most infected tissue only was harvested.
**only upper infected tissue was harvested
N. benthamiana plants were used for the rabbit papillomavirus constructs. Excelsiana plants were chosen for the HPV construct because if this moved forward to a product it would most likely be grown in the field and Excelsiana is a better host for the field. The control virus was wild type TMV U1 for which MD609 plants are the host of choice. Virus is generally allowed to accumulate for longer time periods in the larger MD plants prior to harvest.

Purification of chimeric virus constructs from infected Nicotiana plants Infected plant material was harvested between 8 and 14 days post-inoculation, when the virus accumulation was estimated to be the highest in infected leaf tissues. Only plant material (stem and leaves) above the inoculated leaf was harvested. The harvested tissue was weighed and chopped into small pieces. The virus was extracted by grinding the tissue in a four liter Waring Blender, for two minutes on high speed in a 1:2 ratio (tissue:buffer) of 0.86M sodium chloride, 0.04% sodium metabisulphite solution that had been chilled to 10° C. The temperature of the homogenate (“green juice”) was measured and recorded: this averaged 20.5° C. The homogenate was recovered by squeezing through four layers of cheesecloth, and the volume of homogenate measured. Two 0.5 ml samples of the green juice were collected for analysis by SDS-PAGE, and for bioburden analyses.

The pH of the homogenate was measured and adjusted to pH5.0 with concentrated phosphoric acid. The green juice was then heated to 47° C., and held at that temperature for 15 minutes to coagulate contaminating plant proteins. The homogenate was then cooled to 15° C. in an ice bath. The pH/heat treated homogenate was clarified by centrifugation at 6,000×g for 5 minutes. The supernatant (S1) was decanted through two layers of Miracloth, and the volume of S1 recovered was recorded. Two 0.5 ml samples were collected for SDS-PAGE, protein assay and bioburden analyses. The pellet (P1) was resuspended in distilled water, adjusted to pH 7.4 with NaOH and centrifuged at 6,000×g for 5 minutes to clarify. The volume of the second supernatant (S2) was recorded, and sampled for SDS PAGE to verify that the majority of the virus was in the S1 fraction.

Recombinant virus was precipitated from S1 by adding polyethylene glycol (6000 Da molecular weight) to 4% final concentration. The solution was stirred for 20 minutes, and then chilled on ice for one hour. Precipitated virus was recovered by centrifugation at 10,000×g for 10 minutes. The supernatants were decanted and discarded. The recombinant virus pellets were resuspended in a modified phosphate buffered saline containing 0.86M NaCl, and chilled on ice for 30 minutes. The virus was centrifuged at 8,000×g for 5 minutes to clarify. The supernatants were decanted through miracloth. Two 0.5 ml samples were collected for SDS PAGE analysis. A second PEG-mediated virus precipitation was then performed, as before, and the virus pellets resuspended in phosphate-buffered saline (PBS), pH 7.4. Insoluble material was pelleted by centrifugation at 10,000×g for 5 minutes and the supernatant was recovered with a serological pipette. The final purification step involved freezing and thawing of the virus samples to precipitate any remaining plant contaminants: samples were frozen at −20° C. for several hours and then thawed at room temperature. Insoluble material was eliminated by centrifugation at 10,000×g. The additional freeze-thaw purification steps were not carried out for the TMV:CRPV2.1 and TMV:HPV11L2 samples.

The virus concentration of each fusion was measured using the BCA protein assay with IgG as the standard. Based on the virus concentration determination, a portion of each virus preparation was diluted to 0.5 mg/ml (live virus) or 0.55 mg/ml for the virus inactivation step.

Virus Inactivation with Binary Ethylenimine

Each recombinant TMV preparation was diluted to 0.55 mg/ml in PBS, pH 7.4 to account for the slight dilution due to reagent addition. Virus was chemically inactivated by treatment with binary ethylenimine (BEI), by addition of a 0.1M BEI stock solution to a final concentration of 5 mM BEI. Samples were incubated for 48 hours at 37° C. with constant mixing by rotating tubes end over end in a 37° C. incubator. After 48 hours the BEI was neutralized by addition of a 3 molar excess of sodium thiosulphate.

### EXAMPLE 2

### Viral Hemorrhagic Fever Vaccines

Amongst all of the HFVs, RVFV is perhaps the easiest to weaponize: aerosols are particularly infectious, and have frequently caused infection in laboratory personnel (Borio et al., 2002; Isaacson, 2001). Monoclonal antibody 4D4 has been shown to inhibit RVFV plaque formation in cell culture and to protect mice against lethal challenge (Keegan and Collet, 1986; London et al., 1992).

The general method used in Example 1 was repeated with the linear epitope that binds mAb 4D4 (sequence: KGTMDSGQTKREL) inserted at three different positions in the TMV U1 coat protein: N-terminal (between amino acids 1 and 2); in the surface-located loop structure (between amino acids 64 and 65) and at the C-terminus, between amino acids 155 and 156. The genetic constructs were verified by DNA sequencing, and assigned LSBC identifiers. Table 5 summarizes the expression and MALDI-TOF characterization for these viral fusion constructs.

TABLE 5
RVFV peptide fusions to the TMV U1 coat protein.

| | | Systemic | Soluble | | Actual mass |
|---|---|---|---|---|---|
| Construct | | infection in N. | virions | Theoretical | determined by |
| name | Description | benthamiana | extracted | Mass | MALDI-TOF |
| LSB2472 | 4D4 epitope at | Yes | Yes | | |
| | N-terminus of | | | | |
| | TMV U1 | | | | |
| LSB2471 | 4D4 epitope in | Yes | No | | |
| | surface loop of | | | | |
| | TMV U1 | | | | |
| LSB2470 | 4D4 epitope at | Yes | Yes | | |
| | C-terminus of | | | | |
| | TMV U1 | | | | |

The general method used in Example 1 was repeated with the three known linear epitopes from EBOV GP 1 that bind monoclonal antibodies that neutralize EBOV infection in vitro and in vivo (Wilson et al. 2000). The peptide VYKLDISEA is bound by Mab 6D8-1-2; Mab 13F6-1-2 binds the amino acid sequence DEQHHRRTDND and mAb 12B5-1-1—binds amino acid sequence LITNTIAGV (Wilson et al., 2000). Table 6 summarizes the expression and solubility data for these recombinant TMV virions.

TABLE 6
Solubility and confirmation of three Ebola epitopes
fused to three locations on the TMV U1 coat protein.

| Epitope | | | Predicted | | MALDI | |
|---|---|---|---|---|---|---|
| (sequence¹) | Position² | Solubility | (Da) | Mass | (Da) | mass |
| GP1-393 | N | Yes | 18639 | | 18641 | |
| (VYKLDISEA) | 60's Loop | No | — | | — | |
| | Near C | Yes | 18826 | | 18818 | |
| GP1-405 | N | Yes | 19197 | | 19199 | |
| (DEQHHRRTDND)⁴ | 60's Loop | No | — | | — | |
| | Near C | n.d.³ | — | | — | |
| GP1-481 | N | Yes | 18634 | | 18632 | |
| (LITNTIAGV) | 60's Loop | No | — | | — | |
| | Near C | Yes | 18690 | | 18692 | |

¹This is the minimal consensus sequence.
²N: N-terminus, Near C: the insertion site is before the last four amino acid of the coat protein.
³Not determined.
⁴The extra “D” at the N-terminus was added to the minimal consensus sequence to balance the overall charge of the coat protein.

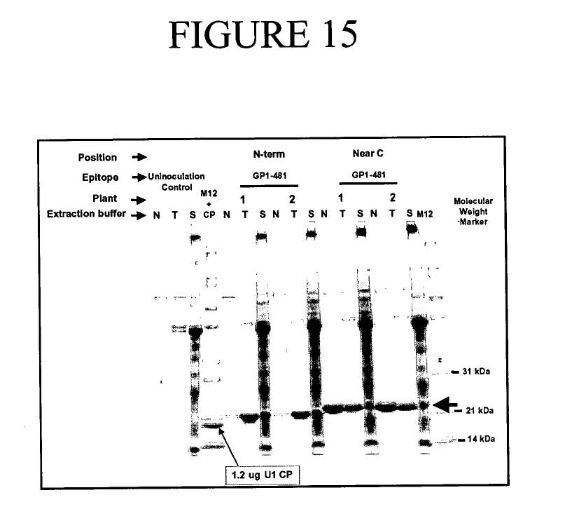

FIG. 15 shows an SDS PAGE gel where extracts from plants infected with infectious transcripts of the various EBOV peptide:TMV fusion constructs were separated according to molecular mass. Proteins from leaf tissues of two infected plants were extracted in sodium acetate “N” buffer (pH 5), the pellet was further extracted in TRIS-C1 “T” buffer (pH 7.5). To extract total protein, another leaf sample was extracted in SDS denaturing “S” buffer (75 mM TRIS (pH 7), 2.5% sodium dodecyl sulfate (SDS), 6% glycerol, 2.5% beta-mecapthoethanol, and 0.05% bromphenol blue). The protein molecular weight marker “M12” is Mark 12 (In vitrogen) spiked with 1.2 mcg of wild type TMV U1 coat protein (CP). The arrow indicates the recombinant product (coat protein fused to an Ebola GP₁ epitope).

### EXAMPLE 3

### Human Immunodeficiency Virus Type 1 (HIV-1) Vaccines

The general method used in Example 1 was repeated with the linear epitopes from HIV proteins. In Table 7, a list of peptides that have been displayed on the surface of TMV U1 and/or U5 virions is displayed.

TABLE 7
HIV-1 Epitopes Expressed on the surface of TMV

| Epitope | | | |
|---|---|---|---|
| Name | Epitope sequence | Location on Env | Comments and (reference) |
| 2F5 short | ELDKWAS | gp41 membrane | Linear epitope. |
| | | proximal region | Induction of Nabs when |
| | | | displayed on the surface |
| | | | of PVX (18); soluble on TMV. |
| 2F5 long | NEQELLELDKWASLWN | gp41 membrane | Identified as being |
| #1 | | proximal region | protected by 2F5 |
| | | | antibody by proteolytic |
| | | | protection assays (46); |
| | | | insoluble on N-terminus |
| | | | of TMV. |
| 2F5 long | EQELLELDKWASLW | gp41 membrane | Selected by 2F5 from a gp160 |
| #2 | | proximal region | expression library (11) |
| 4E10 short | NWFDIT | gp41 membrane | Selected by 4E10 from a |
| | | proximal region | gp160 expression library (11) |
| 4E10 long | LWNWFDITNWLW | gp41 membrane | Core 4E10 recognition site |
| | | proximal region | (11), flanked by adjacent |
| | | | gp41 sequence |
| 2F5/4E10 | LLELDKWASLWNWFDIT | gp41 membrane | Peptide binds both 2F5 |
| | NWLW | proximal region | and 4E10 neutralizing |
| | | | antibodies (11) |
| P195 | KSSGKLISL | gp120 V1 | Identified from phage- |
| | | | displayed peptide |
| | | | library with human |
| | | | HIV-1 antisera (20) |
| P217 | CNGRLYCGP | gp120 C2 | Identified from phage- |
| | | | displayed peptide library |
| | | | with human HIV-1 |
| | | | antisera (20) |
| P197 | GTKLVCFAA | Gp41 | Identified from phage- |
| | | | displayed peptide library |
| | | | with human HIV-1 |
| | | | antisera (20) |
| P287 | CAGGLTCSV | Undetermined | Identified from phage- |
| | | | displayed peptide library |
| | | | with human HIV-1 |
| | | | antisera (20) |
| P335 | SGRLYCHESW | Undetermined | Identified from phage- |
| | | | displayed peptide library |
| | | | with human HIV-1 |
| | | | antisera (20) |
| B2.1 | HERSYMFSDLENRCI | gp120 CD4 | Selected by b12 Mab from |
| | | binding site | phage displayed peptide |
| | | | library. May require display |
| | | | as a homodimer (19) |
| 8.22.2 | TTSIRNKMQKEYALFYK | gp120 V2 region | Linear peptide recognized |
| | | | by Mab isolated from |
| | | | XenoMouse immunized with |
| | | | gp120 (22) |
| TatN | | | |
| terminal | | | |
| B1 | MEPVDPRLEPWKHPGSQP | Tat N terminal | Peptide corresponding |
| | | | to HIV-1 subtype |
| | | | B Tat protein |
| B2 | MEPVDPKLEPWKHPGSQP | Tat N terminal | Peptide corresponding |
| | | | to HIV-1 subtype |
| | | | B Tat protein |
| B3 | MEPVDPNLEPWKHPGSQP | Tat N terminal | Peptide corresponding |
| | | | to HIV-1 subtype |
| | | | B Tat protein |
| C1 | MEPVDPNLEPWKHPGSQP | Tat N terminal | Peptide corresponding |
| | | | to HIV-1 subtype |
| | | | C Tat protein |
| C2 | MDPVDPSLEPWKHPGSQP | Tat N terminal | Peptide corresponding |
| | | | to HIV-1 subtype |
| | | | C Tat protein |
| SA | MEPVDPSLEPWNHPGSQP | Tat N terminal | Peptide corresponding |
| | | | to Tat of HIV-1 |
| | | | subtype found in Nigeria |
| Tat | | | |
| Peptide 3 | | | |
| B1 | PTSQSRGDPTGPKE | Tat cellular | Peptide corresponding |
| | | binding domain | to HIV-1 subtype B |
| | | | Tat protein |
| B2 | PSSQPRGDPTGPKE | Tat cellular | Peptide corresponding |
| | | binding domain | to HIV-1 subtype B |
| | | | Tat protein |
| B3 | PASQSRGDPTGPTE | Tat cellular | Peptide corresponding |
| | | binding domain | to HIV-1 subtype B |
| | | | Tat protein |
| C1 | PLPRTQGDPTGSEE | Tat cellular | Peptide corresponding |
| | | binding domain | to HIV-1 subtype C |
| | | | Tat protein |
| C2 | PLPQTRGDPTGSKE | Tat cellular | Peptide corresponding |
| | | binding domain | to HIV-1 subtype C |
| | | | Tat protein |
| SA | PLPTTRGNPTGPKE | Tat cellular | Peptide corresponding |
| | | binding domain | to HIV-1 subtype C |
| | | | Tat protein |
| Gp41 | | | |
| | LQARILAVE | Gp41 | Peptide from gp41 |
| | | | of an HIV-1 |
| | | | subtype B strain |
| SA | LQARVLAVEGQARVLALER | Gp41 | Peptide from gp41 |
| | | | of an HIV-1 type |
| | | | found in Nigeria |
| SAELD | EKNEQDLLALDKWASLWN | Gp41 | Peptide from gp41 |
| | | | of an HIV-1 type |
| | | | found in Nigeria |
| V3 Loop | | | |
| V3MN | ADTIGPGRAFYTTK | Gp120 V3 loop | Peptide from crown |
| | | | of the V3 loop of |
| | | | TCLA HIV-1 strain MN |
| V3BaL | ADTIGPGRAFYTTG | Gp120 V3 loop | Peptide from crown |
| | | | of the V3 loop of |
| | | | HIV-1 strain BaL |
| Nigeria | ADTIGPGQAFYAGG | Gp120 V3 loop | Peptide from crown |
| | | | of the V3 loop of |
| | | | HIV-1 strain |
| | | | found in Nigeria |

The expression, extraction and solubility data for these recombinant viruses is summarized in Table 8 below.

TABLE 8

| | | | | Epitope Peptide | | Charge | Charge | |
|---|---|---|---|---|---|---|---|---|
| pLSB# | | | Name | Sequence | PI | @pH5 | @pH7 | Acetate |
| | U5 | — | TMV | | | | | |
| | U1 | — | TMV | | | | | |
| 2405 | U1 | N | 16L2 | GLVEETSFIDAGAP | 4.41 | −3.7 | −5.1 | |
| 2405 | U1 | N | 16L2 | GLVEETSFIDAGAP | 4.41 | −3.7 | −5.1 | \ |
| 2409 | U5 | N | 16L2 | GLVEETSFIDAGAP | 4.41 | −3.7 | −5.1 | |
| 2409 | U5 | N | 16L2 | GLVEETSFIDAGAP | 4.41 | −3.7 | −5.1 | \ |
| 2278 | U1 | C | 16L2C | LVEETSFIDAGAP | 4.41 | −3.7 | −5.1 | + |
| 2278 | U1 | C | 16L2C | LVEETSFIDAGAP | 4.41 | −3.7 | −5.1 | + |
| 2402 | U1 | N | 18L2 | GLIEDSSVVTSGAP | 4.50 | −2.8 | −4.1 | |
| 2402 | U1 | N | 18L2 | GLIEDSSVVTSGAP | 4.50 | −2.8 | −4.1 | \ |
| 2416 | U5 | N | 18L2 | GLIEDSSVVTSGAP | 4.50 | −2.8 | −4.1 | |
| 2416 | U5 | N | 18L2 | GLIEDSSWTSGAP | 4.50 | −2.8 | −4.1 | \ |
| 2270 | U1 | C | 18L2C | LIEDSSVVTSGAP | 4.50 | −2.8 | −4.1 | |
| 2281 | U1 | C | 18L2C | LIEDSSVVTSGAP | 4.50 | −2.8 | −4.1 | + |
| 2281 | U1 | C | 18L2C | LIEDSSVVTSGAP | 4.50 | −2.8 | −4.1 | + |
| 2427 | U1 | C | 4E10 | NWFDIT | 4.82 | −1.0 | −2.1 | − |
| 2430 | U1 | L | 4E10 | NWFDIT | 4.82 | −1.0 | −2.1 | − |
| | U1 | L | 4E10 | NWFDIT | 4.82 | −1.0 | −2.1 | − |
| 2407 | U1 | N | 4E10 | GNWFDIT | 4.82 | −1.0 | −2.1 | na |
| 2407 | U1 | N | 4E10 | GNWFDIT | 4.82 | −1.0 | −2.1 | \ |
| 2411 | U5 | N | 4E10 | GNWFDIT | 4.82 | −1.0 | −2.1 | na |
| 2411 | U5 | N | 4E10 | GNWFDIT | 4.82 | −1.0 | −2.1 | \ |
| 2406 | U1 | N | 6/11L2 | GLIEESAIINAGAP | 4.54 | −2.8 | −4.1 | |
| 2406 | U1 | N | 6/11L2 | GLIEESAIINAGAP | 4.54 | −2.8 | −4.1 | \ |
| 2282 | U1 | C | 6/11L2C | LIEESAIINAGAP | 4.54 | −2.8 | −4.1 | + |
| 2282 | U1 | C | 6/11L2C | LIEESAIINAGAP | 4.54 | −2.8 | −4.1 | + |
| 2414 | U5 | N | 6/11L2C | GLIEESAIINAGAP | 4.54 | −2.8 | −4.1 | |
| 2414 | U5 | N | 6/11L2C | GLIEESAIINAGAP | 4.54 | −2.8 | −4.1 | \ |
| 2417 | U1 | N | CRPVL2.1 | GVGPLDIVPEVADPGGPTL | 4.38 | −3.8 | −5.1 | |
| 2419 | U1 | N | CRPVL2.1 | GVGPLDIVPEVADPGGPTL | 4.38 | −3.8 | −5.1 | |
| 2283 | U1 | C | CRPVL2.1C | VGPLDIVPEVADPGGPTL | 4.69 | −1.8 | −4.0 | + |
| 2283 | U1 | C | CRPVL2.1C | VGPLDIVPEVADPGGPTL | 4.69 | −1.8 | −4.0 | + |
| 2274 | U1 | L | CRPVL2.1L | VGPLDIVPEVADPGGPTL | 4.50 | −2.8 | −4.1 | ˜ |
| 2420 | U1 | N | CRPVL2.2 | GPGGPTLVSLHELPAETP | 4.69 | −1.8 | −4.0 | |
| 2410 | U5 | N | CRPVL2.2 | GPGGPTLVSLHELPAETP | 4.69 | −1.8 | −4.0 | |
| 2288 | U1 | C | CRPVL2.2C | PGGPTLVSLHELPAETPY | 4.69 | −1.8 | −4.0 | + |
| 2287 | U1 | L | CRPVL2.2L | PGGPTLVSLHELPAETPY | 4.89 | −0.9 | −3.0 | + |
| 2287 | U1 | L | CRPVL2.2L | PGGPTLVSLHELPAETPY | 4.89 | −0.9 | −3.0 | − |
| 2461 | U1 | C | E7CTL | DRAHYNIVTFAG | 5.00 | 0.0 | −1.9 | na |
| 2463 | U1 | C | E7CTL | DRAHYNIVTFAG | 5.00 | 0.0 | −1.9 | − |
| 2462 | U1 | L | E7CTL | DRAHYNIVTFAG | 5.65 | 1.0 | −0.9 | − |
| 2462 | U1 | L | E7CTL | DRAHYNIVTFAG | 5.65 | 1.0 | −0.9 | na |
| 2461 | U1 | N | E7CTL | GDRAHYNIVTFAG | 5.00 | 0.0 | −1.9 | − |
| 2463 | U1 | N | E7CTL | GDRAHYNIVTFAG | 5.00 | 0.0 | −1.9 | na |
| 2463 | U1 | N | E7CTL | GDRAHYNIVTFAG | 5.00 | 0.0 | −1.9 | |
| 2619 | U5 | C | E7CTL | AMDRAHYNIVTFAG | 4.55 | −1.3 | −2.9 | − |
| 2428 | U1 | C | E7CTLTH | DRAHYNIVTFAG | 5.16 | 0.1 | −2.0 | + |
| 2275 | U1 | L | E7CTLTH | QAEPDRAHYNIVTF | 5.16 | 0.1 | −2.0 | − |
| 2403 | U1 | N | E7CTLTH | GQAEPDRAHYNIVTF | 5.16 | 0.1 | −2.0 | |
| 2290 | U1 | C | E7CTLTHPL | QAEPDRAHYNIVTFCCKCD | 5.70 | 1.1 | −1.2 | + |
| 2276 | U1 | L | E7CTLTHPL | QAEPDRAHYNIVTFCCKCD | 5.70 | 1.1 | −1.2 | − |
| 2400 | U1 | N | E7CTLTHPL | GQAEPDRAHYNIVTFCCKCD | 5.70 | 1.1 | −1.2 | |
| 2289 | U1 | C | ELDKWAS | ELDKWAS | 4.87 | −0.9 | −2.1 | + |
| 2277 | U1 | L | ELDKWAS | ELDKWAS | 4.87 | −0.9 | −2.1 | + |
| 2013 | U1 | N | ELDKWAS | GELDKWAS | 4.87 | −0.9 | −2.1 | + |
| 2013 | U1 | N | ELDKWAS | GELDKWAS | 4.87 | −0.9 | −2.1 | na |
| 2013 | U1 | N | ELDKWAS | GELDKWAS | 4.87 | −0.9 | −2.1 | \ |
| 2413 | U5 | N | ELDKWAS | GELDKWAS | 4.87 | −0.9 | −2.1 | na |
| 2413 | U5 | N | ELDKWAS | GELDKWAS | 4.87 | −0.9 | −2.1 | \ |
| 2429 | U1 | C | EQ | EQELLELDKWASLW | 4.59 | −2.7 | −4.1 | − |
| 2271 | U1 | L | EQ | EQELLELDKWASLW | 4.59 | −2.7 | −4.1 | − |
| 2401 | U1 | N | EQ | GEQELLELDKWASLW | 4.59 | −2.7 | −4.1 | na |
| 2401 | U1 | N | EQ | GEQELLELDKWASLW | 4.59 | −2.7 | −4.1 | \ |
| 2412 | U5 | N | EQ | GEQELLELDKWASLW | 4.59 | −2.7 | −4.1 | na |
| 2412 | U5 | N | EQ | GEQELLELDKWASLW | 4.59 | −2.7 | −4.1 | \ |
| 3431 | U1 | L | L1I23 | QPLGVGISGHPLLNKLDDTE | 4.68 | −1.9 | −4.0 | − |
| 2418 | U1 | N | L1I23 | GQPLGVGISGHPLLNKLDDTE | 4.68 | −1.9 | −4.0 | |
| 2279 | U1 | C | L1I23C | QPLGVGISGHPLLNKLDDTE | 4.68 | −1.9 | −4.0 | + |
| 2286 | U1 | C | L1I23C | QPLGVGISGHPLLNKLDDTE | 4.68 | −1.9 | −4.0 | − |
| | U1 | L | L1I23L | QPLGVGISGHPLLNKLDDTE | 4.87 | −0.9 | −3.0 | |
| 2404 | U1 | N | L1J4 | GGENVPDDLYIKGSGS | 4.50 | −2.8 | −4.1 | |
| 2284 | U1 | C | L1J4C | GENVPDDLYIKGSGS | 4.50 | −2.8 | −4.1 | + |
| 2466 | U1 | C | LQ | MLQARILAVEAGA | 4.75 | −0.9 | −2.0 | + |
| 2466 | U1 | C | LQ | MLQARILAVEAGA | 4.75 | −0.9 | −2.0 | + |
| 2465 | U1 | L | LQ | GSPMLQARILAVEAGAGPS | 5.05 | 0.1 | −1.0 | − |
| 2465 | U1 | L | LQ | GSPMLQARILAVEAGAGPS | 5.05 | 0.1 | −1.0 | − |
| 2464 | U1 | N | LQ | LQARILAVEAGA | 4.75 | −0.9 | −2.0 | − |
| 2464 | U1 | N | LQ | LQARILAVEAGA | 4.75 | −0.9 | −2.0 | + |
| 2467 | U1 | C | LQN | MLQARVLAVEGQARVLALEAGA | 4.75 | −0.8 | −2.0 | − |
| 2467 | U1 | C | LQN | MLQARVLAVEGQARVLALEAGA | 4.75 | −0.8 | −2.0 | na |
| 2468 | U1 | L | LQN | GSPMLQARVLAVEGQARVLALEAGAGPS | 5.05 | 0.2 | −1.0 | − |
| 2468 | U1 | L | LQN | GSPMLQARVLAVEGQARVLALEAGAGPS | 5.05 | 0.2 | −1.0 | − |
| 2469 | U1 | N | LQN | LQARVLAVEGQARVLALEAGA | 4.75 | −0.8 | −2.0 | − |
| 2469 | U1 | N | LQN | LQARVLAVEGQARVLALEAGA | 4.75 | −0.8 | −2.0 | na |
| 2421 | U1 | N | ROPVL2.1 | GVGPLEVIPEAVDPAGSSI | 4.45 | −3.7 | −5.0 | |
| 2422 | U1 | N | ROPVL2.1 | GPAGSSIVPLEEYPAEIP | 4.45 | −3.7 | −5.0 | |
| 2415 | U5 | N | ROPVL2.1 | GVGPLEVIPEAVDPAGSSI | 4.15 | −5.0 | −6.0 | |
| 2285 | U1 | C | ROPVL2.1C | VGPLEVIPEAVDPAGSSI | 4.41 | −3.7 | −5.1 | + |
| 2285 | U1 | C | ROPVL2.1C | VGPLEVIPEAVDPAGSSI | 4.41 | −3.7 | −5.1 | + |
| 2272 | U1 | L | ROPVL2.1L | VGPLEVIPEAVDPAGSSI | 4.54 | −2.8 | −4.1 | − |
| 2280 | U1 | C | ROPVL2.2C | PAGSSIVPLEEYPAEIPT | 4.45 | −3.7 | −5.1 | + |
| 2280 | U1 | C | ROPVL2.2C | PAGSSIVPLEEYPAEIPT | 4.45 | −3.7 | −5.1 | + |
| 2273 | U1 | L | ROPVL2.2L | PAGSSIVPLEEYPAEIPT | 4.57 | −2.7 | −4.1 | − |
| 2408 | U5 | N | ROPVL2.2N | GPAGSSIVPLEEYPAEIP | 4.25 | −5.0 | −6.0 | |
| 2470 | U1 | C | RVFV | MYKGTMDSGQTKREAGA | 5.05 | 0.2 | −1.0 | + |
| 2470 | U1 | C | RVFV | MYKGTMDSGQTKREAGA | 5.05 | 0.2 | −1.0 | + |
| 2471 | U1 | L | RVFV | GSPMYKGTMDSGQTKREAGAGPS | 7.00 | 1.1 | 0.0 | − |
| 2471 | U1 | L | RVFV | GSPMYKGTMDSGQTKREAGAGPS | 7.00 | 1.1 | 0.0 | − |
| 2472 | U1 | N | RVFV | YKGTMDSGQTKREAGA | 5.05 | 0.2 | −1.0 | − |
| 2472 | U1 | N | RVFV | YKGTMDSGQTKREAGA | 5.05 | 0.2 | −1.0 | − |
| 2475 | U1 | C | TAT1A | MEPVDPRLEPWKHPGSQAGA | 4.52 | −2.8 | −4.9 | + |
| 2475 | U1 | C | TAT1A | MEPVDPRLEPWKHPGSQAGA | 4.52 | −2.8 | −4.9 | − |
| 2474 | U1 | L | TAT1A | GSPMEPVDPRLEPWKHPGSQAGAGPS | 4.62 | −1.8 | −3.9 | − |
| 2474 | U1 | L | TAT1A | GSPMEPVDPRLEPWKHPGSQAGAGPS | 4.62 | −1.8 | −3.9 | * |
| 2473 | U1 | N | TAT1A | EPVDPRLEPWKHPGSQAGA | 4.52 | −2.8 | −4.9 | + |
| 2473 | U1 | N | TAT1A | EPVDPRLEPWKHPGSQAGA | 4.52 | −2.8 | −4.9 | + |
| 2478 | U1 | C | TAT1B | MEPVDPSLEPWNHPGSQAGA | 4.75 | −0.8 | −2.9 | − |
| 2478 | U1 | C | TAT1B | MEPVDPSLEPWNHPGSQAGA | 4.77 | −0.8 | −2.9 | − |
| 2477 | U1 | L | TAT1B | GSPMEPVDPSLEPWNHPGSQAGAGPS | 5.05 | 0.2 | −1.9 | − |
| 2477 | U1 | L | TAT1B | GSPMEPVDPSLEPWNHPGSQAGAGPS | 5.05 | 0.2 | −1.9 | − |
| 2476 | U1 | N | TAT1B | EPVDPSLEPWNHPGSQAGA | 4.82 | −0.8 | −2.9 | − |
| 2476 | U1 | N | TAT1B | EPVDPSLEPWNHPGSQAGA | 4.82 | −0.8 | −2.9 | + |
| 2481 | U1 | C | TAT3B | MPTSQSRGDPTGPKEAGA | 4.77 | −0.8 | −2.0 | + |
| 2481 | U1 | C | TAT3B | MPTSQSRGDPTGPKEAGA | 4.77 | −0.8 | −2.0 | + |
| 2480 | U1 | L | TAT3B | GSPMPTSQSRGDPTGPKEAGAGPS | 5.05 | 0.1 | −1.0 | − |
| 2480 | U1 | L | TAT3B | GSPMPTSQSRGDPTGPKEAGAGPS | 5.05 | 0.1 | −1.0 | + |
| 2479 | U1 | N | TAT3B | PTSQSRGDPTGPKEAGA | 4.77 | −0.8 | −2.0 | + |
| 2479 | U1 | N | TAT3B | PTSQSRGDPTGPKEAGA | 4.77 | −0.8 | −2.0 | + |
| 2484 | U1 | C | TAT3N | MPLPTTRGNPTGPKEAGA | 4.05 | 0.1 | −1.0 | + |
| 2484 | U1 | C | TAT3N | MPLPTTRGNPTGPKEAGA | 4.05 | 0.1 | −1.0 | + |
| 2483 | U1 | L | TAT3N | GSPMPLPTTRGNPTGPKEAGAGPS | 6.80 | 1.1 | 0.0 | \ |
| 2483 | U1 | L | TAT3N | GSPMPLPTTRGNPTGPKEAGAGPS | 6.80 | 1.1 | 0.0 | − |
| 2482 | U1 | N | TAT3N | PLPTTRGNPTGPKEAGA | 5.05 | 0.1 | −1.0 | \ |
| 2482 | U1 | N | TAT3N | PLPTTRGNPTGPKEAGA | 5.05 | 0.1 | −1.0 | na |
| | U1 | N | V3 | GCTRPNYNKRKRIHIGPGRAFYTTKNIIGTIRQAHC | 9.67 | 8.9 | 6.1 | |
| 2485 | U1 | C | V3BAL | MIGPGRAFYTTGAGA | 5.02 | 0.0 | −1.0 | na |
| 2487 | U1 | C | V3BAL | MIGPGRAFYTTGAGA | 5.02 | 0.0 | −1.0 | − |
| 2486 | U1 | L | V3BAL | GSPMIGPGRAFYTTGAGAGPS | 6.80 | 1.0 | 0.0 | − |
| 2486 | U1 | L | V3BAL | GSPMIGPGRAFYTTGAGAGPS | 6.80 | 1.0 | 0.0 | na |
| 2485 | U1 | N | V3BAL | IGPGRAFYTTGAGA | 5.02 | 0.0 | −1.0 | + |
| 2487 | U1 | N | V3BAL | IGPGRAFYTTGAGA | 5.02 | 0.0 | −1.0 | na |
| 2488 | U1 | C | V3MN | MIGPGRAFYTTKAGA | 6.80 | 1.0 | 0.0 | na |
| 2490 | U1 | C | V3MN | MIGPGRAFYTTKAGA | 6.80 | 1.0 | 0.0 | − |
| 2489 | U1 | L | V3MN | GSPMIGPGRAFYTTKAGAGPS | 9.30 | 2.0 | 1.0 | − |
| 2489 | U1 | L | V3MN | GSPMIGPGRAFYTTKAGAGPS | 9.30 | 2.0 | 1.0 | − |
| 2488 | U1 | N | V3MN | IGPGRAFYTTKAGA | 6.80 | 1.0 | 0.0 | \ |
| 2490 | U1 | N | V3MN | IGPGRAFYTTKAGA | 6.80 | 1.0 | 0.0 | na |
| 2493 | U1 | C | V3NIG | MIGPGQAFYAGGAGA | 4.72 | −1.0 | −2.0 | − |
| 2493 | U1 | C | V3NIG | MIGPGQAFYAGGAGA | 4.72 | −1.0 | −2.0 | + |
| 2492 | U1 | L | V3NIG | GDPMIGPGQAFYAGGAGAGPS | 5.00 | 0.0 | −1.0 | − |
| 2492 | U1 | L | V3NIG | GSPMIGPGQAFYAGGAGAGPS | 5.00 | 0.0 | −1.0 | − |
| 2491 | U1 | N | V3NIG | IGPGQAFYAGGAGA | 4.70 | −1.0 | −2.0 | − |
| 2491 | U1 | N | V3NIG | IGPGQAFYAGGAGA | 4.70 | −1.0 | −2.0 | + |

| | | | | | Age at | | | |
|---|---|---|---|---|---|---|---|---|
| | Tris | | | | inoculation | | Plant | Soluble |
| pLSB# | pH7 | SDS | S1 | S2 | (Days) | DPI | Score | Virus |
| 2405 | | | | | | | | |
| 2405 | + | + | − | + | 28 | 7 | SI | Y |
| 2409 | | | | | | | | |
| 2409 | + | + | + | + | 28 | 7 | SI | Y |
| 2278 | na | + | + | + | 23 | 8 | SI | Y |
| 2278 | + | + | + | + | 26 | 9 | SI, C | |
| 2402 | | | | | | | | |
| 2402 | + | + | + | − | 28 | 7 | U | Y |
| 2416 | | | | | | | | |
| 2416 | + | + | + | + | 28 | 7 | SI | Y |
| 2270 | | | | | − | | | N |
| 2281 | na | + | + | + | 21 | 11 | SI | Y |
| 2281 | + | + | + | + | 26 | 9 | SI, C | Y |
| 2427 | − | − | − | − | 22 | 13 | U | N |
| 2430 | − | + | − | − | 22 | 13 | N | N |
| | − | − | − | − | | | | Y |
| 2407 | − | − | − | − | 24 | 21 | N, SI | N |
| 2407 | − | + | − | − | 28 | 7 | U | N |
| 2411 | + | + | + | + | 24 | 21 | N, SI | Y |
| 2411 | + | + | + | + | 28 | 7 | SI | Y |
| 2406 | | | | | | | | |
| 2406 | + | + | + | + | 28 | 7 | SI | Y |
| 2282 | na | + | + | + | 21 | 11 | SI | Y |
| 2282 | + | + | + | + | 26 | 9 | SI, C | Y |
| 2414 | | | | | | | | |
| 2414 | + | − | + | − | 28 | 7 | U, N | Y |
| 2417 | | | | | − | | | Y |
| 2419 | | | | | | | | |
| 2283 | na | + | + | + | 21 | 11 | SI | Y |
| 2283 | + | + | + | + | 26 | 9 | SI, C | Y |
| 2274 | na | ˜ | ˜ | ˜ | 23 | 8 | SI, N | N |
| 2420 | | | | | − | | | Y |
| 2410 | | | | | | | | |
| 2288 | + | + | + | + | 24 | | | Y |
| 2287 | + | + | + | + | 24 | | | N |
| 2287 | − | − | − | − | 26 | 9 | N | N |
| 2461 | na | na | ? | + | 25 | 9 | | Y |
| 2463 | + | + | − | − | 24 | 14 | SI | |
| 2462 | + | + | − | + | 27 | 13 | SI | |
| 2462 | na | na | ? | + | 25 | 9 | | Y |
| 2461 | + | + | + | + | 26 | 13 | SI | |
| 2463 | na | na | ? | + | 25 | 9 | | Y |
| 2463 | ++ | na | − | + | | | S | |
| 2619 | − | na | ? | − | | | S | N |
| 2428 | + | + | + | + | 22 | 13 | SI, C | Y |
| 2275 | na | − | − | − | 23 | 8 | W, SN | N |
| 2403 | | | | | | | | |
| 2290 | + | + | + | + | 25 | 9 | | Y |
| 2276 | na | − | − | − | 23 | 8 | N, M, SI | N |
| 2400 | | | | | | | | |
| 2289 | + | + | + | + | 25 | 9 | | Y |
| 2277 | + | + | + | + | 24 | | | Y |
| 2013 | na | + | + | + | 21 | 11 | SI | Y |
| 2013 | Mixed | | | | 24 | 21 | SI | Y |
| | Up | | | | | | | |
| 2013 | + | + | + | + | 28 | 7 | SI | Y |
| 2413 | Mixed | | | | 24 | 21 | SI | N |
| | up | | | | | | | |
| 2413 | + | + | + | − | 28 | 7 | U | Y |
| 2429 | − | − | − | − | 22 | 13 | U | N |
| 2271 | na | − | − | − | 23 | 8 | M, LSI | N |
| 2401 | | | | | 24 | 21 | SI | N |
| 2401 | + | − | − | − | 28 | 7 | U | N |
| 2412 | | | | | 24 | 21 | SI | Y |
| 2412 | − | − | − | − | 28 | 7 | U | N |
| 3431 | − | + | − | − | 22 | 13 | R | N |
| 2418 | | | | | − | | | Y |
| 2279 | na | + | + | + | 23 | 8 | SI | Y |
| 2286 | − | − | − | − | 24 | | | Y |
| 2404 | | | | | | | | |
| 2284 | na | + | + | + | 21 | 11 | SI | Y |
| 2466 | + | + | + | + | 24 | 14 | SI | |
| 2466 | − | na | ? | na | 24 | | SI | |
| 2465 | + | + | − | + | 27 | 13 | SI | |
| 2465 | − | na | − | na | 24 | | SI | |
| 2464 | + | + | − | + | 26 | 13 | SI | |
| 2464 | − | na | + | + | 24 | | SI | |
| 2467 | + | + | − | + | 25 | 9 | | Y |
| 2467 | na | na | na | na | 25 | | N | |
| 2468 | + | + | + | + | 25 | 9 | | Y |
| 2468 | − | na | − | − | 25 | | SI | |
| 2469 | + | + | − | + | 25 | 9 | | Y |
| 2469 | na | na | na | na | 25 | | N | |
| 2421 | | | | | − | | | Y |
| 2422 | | | | | | | | |
| 2415 | | | | | | | | |
| 2285 | na | + | + | + | 21 | 11 | SI | Y |
| 2285 | + | + | + | + | 26 | 9 | SI, C | Y |
| 2272 | na | − | − | − | 23 | 8 | N, SI | N |
| 2280 | na | + | + | + | 23 | 8 | SI | Y |
| 2280 | + | + | + | + | 26 | 9 | SI, C | Y |
| 2273 | na | − | − | − | 23 | 8 | N, LSI | N |
| 2408 | | | | | | | | |
| 2470 | + | + | + | + | 25 | 9 | | Y |
| 2470 | + | na | + | + | 24 | | S | |
| 2471 | + | + | − | + | 25 | 9 | | Y |
| 2471 | + | na | − | − | 24 | | S | |
| 2472 | + | + | − | − | 25 | 9 | | N |
| 2472 | + | na | + | + | 24 | | S | |
| 2475 | + | + | + | + | 24 | 14 | SI | |
| 2475 | + | na | ? | − | 24 | | SI | |
| 2474 | − | + | − | − | 27 | 13 | IN, 1SI | |
| 2474 | + | na | + | na | 24 | | SI | |
| 2473 | + | + | + | + | 26 | 13 | SI | |
| 2473 | + | na | + | na | 24 | | SI | |
| 2478 | + | + | − | + | 24 | 14 | SI | |
| 2478 | + | na | + | + | 27 | | SI | |
| 2477 | + | + | + | + | 27 | 13 | SI | |
| 2477 | na | na | ? | + | 27 | | SI | |
| 2476 | + | + | − | + | 26 | 13 | SI | |
| 2476 | na | na | ? | + | 27 | | SI | |
| 2481 | + | + | + | + | 24 | 14 | SI | |
| 2481 | na | na | + | + | 25 | | SI | |
| 2480 | − | + | − | − | 27 | 13 | SI | |
| 2480 | + | na | ? | + | 25 | | SI | |
| 2479 | − | + | + | − | 26 | 13 | SI | |
| 2479 | + | na | + | na | 25 | | SI | |
| 2484 | + | + | + | + | 24 | 14 | SI | |
| 2484 | + | na | + | + | 23 | | SI | |
| 2483 | \ | \ | \ | \ | 27 | 13 | N | |
| 2483 | − | na | − | − | 23 | | SI | |
| 2482 | \ | \ | \ | \ | 26 | 13 | N | |
| 2482 | na | na | na | na | 23 | | N | |
| 2485 | na | na | | − | 25 | 8 | S | |
| 2487 | + | + | − | + | 24 | 14 | SI | |
| 2486 | + | + | − | + | 27 | 13 | SI | |
| 2486 | na | na | | + | 25 | 8 | S | |
| 2485 | + | + | + | + | 26 | 13 | SI | |
| 2487 | na | na | | + | 25 | 8 | S | |
| 2488 | na | na | na | na | 24 | | N | |
| 2490 | + | + | − | + | 24 | 14 | SI | |
| 2489 | − | + | − | − | 27 | 13 | 1N, 1SI | |
| 2489 | + | na | ? | ? | 24 | | S, N | |
| 2488 | \ | \ | \ | \ | 26 | 13 | N | |
| 2490 | na | na | na | na | 24 | | N | |
| 2493 | + | + | − | + | 24 | 14 | 1N | |
| 2493 | + | na | − | + | 24 | 9 | SI | |
| 2492 | − | + | − | − | 27 | 13 | SI | |
| 2492 | − | na | + | − | 24 | 9 | SI | |
| 2491 | + | + | + | + | 26 | 13 | SI | |
| 2491 | + | na | .+ | + | 24 | 9 | SI | |

### EXAMPLE 4

### Parvo Virus Vaccines

The general method used in Example 1 was repeated with the linear epitopes from parvo virus. The N-terminus of FPV, CPV and PPV VP2 contains a major neutralizing determinant for the virus; this is a linear epitope, present in the first 23 amino acids of the protein. Neutralizing antibodies may be induced in animals immunized with peptides derived from the first 23 amino acids of VP2 (Langeveld et al., 1995; 2001). The sequence of the N-terminus of VP2 follows: MSDGAVQPDGGQPAVRNERATGS.

We designed a synthetic DNA sequences which would encode various portions of the N-terminal VP2 sequence. The synthetic DNA was synthesized in complementary oligonucleotides, and inserted into the coat protein of TMV U1 and TMV U5. These sequences of the peptides were denoted Parvol; Parvo2; and Parvo3. The amino acid sequences of these peptides are as follows:

| | | |
|---|---|---|
| Parvo1: | MSDGAVQPDGGQPAVRNERAT | (21 amino acids) |
| Parvo2: | MSDGAVQPDGGQPAVRNERA | (20 amino acids) |
| Parvo3: | VQPDGGQPAVRNERAT | (16 amino acids) |

### EXAMPLE 5

### Determination of Viral Infectivity and Bacterial Bioburden of Recombinant TMV Particles Carrying Vaccine Epitopes

A list of final products with titers diluents, carrier are given in Table 3.

TABLE 3
Papillomavirus Vaccines Final Volumes and Virus Quantities

| | | | | Volume | | | Concentrate |
|---|---|---|---|---|---|---|---|
| | | virus | Total | used for | | # Vials | Vol. |
| | BCA IgG | volume | Virus | dilutions | # Vials | BEI | @−20oC |
| Virus Name | (mg/mL) | (mL) | (mg) | (mL) | UT | treated | (mL) |
| TMV:CRPV2.1 | 15.5 | 11.2 | 173.6 | 3.2 | 47 | 45 | 7 |
| TMV:CRPV2.1 Alt | 19.6 | 7 | 137.2 | 2 | 37 | 35 | 4 |
| TMV:HPV-11 L2 | 28.1 | 35 | 983.5 | 3 | 60 | 60 | 32 |
| TMV:ROPV2.2 | 6.4 | 57 | 364.8 | 8 | 50 | 0* | 46 |
| TMV:ROPV2.2 F/T | 5.3 | 20 | 106 | 9.5 | 49 | 49 | 10 |
| TMV wild type | 16 | 21 | 336 | 4.5 | 48 | 68** | 15.5 |
| F/T | | | | | | | |
| TMV:CRPV2.2 F/T | 4.6 | 50 | 230 | 11.2 | 50 | 51 | 38 |
| TMV:ROPV2.1 | 19.3 | 50 | 965 | 2.65 | 50 | 50 | 46 |
| F/T 4/16/03 | | | | | | | |

*frozen as bulk (46 mL)
**froze 24 mL as bulk
F/T = freeze thaw

Process samples and final product for bacterial bioburden were monitored by aseptically plating 10 μl or 100 μl samples on bacterial nutrient agar in a laminar flow hood. Plates were inverted and incubated at room temperature for four days. The bacterial colony counts were recorded after four days. The plates were then transferred to a 33° C. incubator for a further four days, and bacterial colony counts were recorded again. Bioburden assays for final fill samples were run in duplicate and the results averaged. Bioburden decreased with each sequential processing step from 420-3800 colony forming units (CFU) per ml in the initial homogenate, to 0-130 CFU/ml in the final (concentrated) virus preparations. The final dilute vaccines had no detectable bioburden in either the untreated or BEI-treated samples.

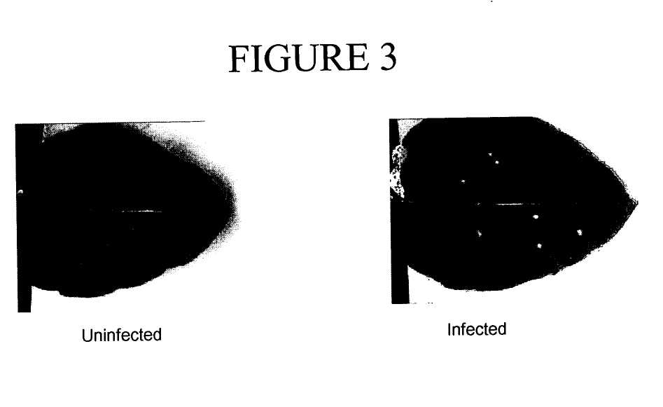

TMV infectivity was determined using a local lesion host Nicotiana tabacum var. Xanthi, cultivar “Glurk”. This assay is accepted by the United States Department of Agriculture as a method for evaluating tobacco mosaic virus infectivity. The limit of detection for the Glurk assay is 10 pg/μl. Glurk plants were sown into flats and transplanted into 3.5 inch pots at two weeks post sowing. The Glurks were prepared for inoculation by numbering the leaves to be inoculated with a lab marker on the upper distal portion of the leaf. A small amount of silicon carbide (400 mesh) was sprinkled on each numbered leaf. One hundred microliters of the sample to be assayed was dispensed onto the upper surface of the appropriate leaf and gently spread over the entire surface of the leaf. Glurk plants were scored 4 to 6 days post-inoculation by counting the number of local lesions that had formed on the leaf surface (see FIG. 3). The Glurk local lesion assays were run in triplicate and the results were averaged. The infectivity of the final vialed vaccine products is summarized in Table 4; where the average number of local lesions for the 10⁻³ dilution was used to derive the infectivity measurement.

TABLE 4
Infectivity of TMV: papillomavirus epitope vaccines

| | | Number of | Number of |
|---|---|---|---|
| | | Local Lesions | Local Lesions |
| | | per ml in | per ml in the |
| Construct | | the Untreated | BEI-treated |
| Name | Virus Name | Vaccine Sample | Vaccine Sample |
| pLSB2283 | TMV:CRPV2.1 | 2.71 × 10⁶ | 0 |
| pLSB2288 | TMV:CRPV2.2 | 1.52 × 10⁶ | 0 |
| pLSB2285 | TMV:ROPV2.1 | 4.0 × 10⁵ | 0 |
| pLSB2280 | TMV:ROPV2.2 | Tntc; | 10 |
| | | 1.0 × 10⁷* | |
| pLSB2282 | TMV:HPV-11 L2 | 5.6 × 10⁵ | 0 |
| Wild type | TMV | 1.37 × 10⁶ | 0 |
| TMV | | | |

*figure derived from 10⁻⁴ dilution; for all other assays the results from the 10⁻³ dilution point is depicted.

These results demonstrate that treatment with BEI is an effective means for inactivation of the infectivity of tobacco mosaic virus vaccines.

All of the vaccine products were analyzed for endotoxin using the Associates of Cape Cod gel clot assay. Additional release testing was done on all of the final vaccine preparations, which included concentration determination by BCA assay, as well as amino acid analysis by post column derivitization, SDS-PAGE for purity assessment and concentration, molecular weight determination by MALDI-TOF, tryptic MALDI-TOF if required, pH and appearance.

There was no endotoxin detected in any of the BEI-treated samples after testing multiple dilutions of the samples. Low levels of endotoxin were present in the TMV:HPV11L2 (1 EU/dose), TMV:ROPV2.1 (2 EU/dose) and TMV:ROPV2.2 (2 EU/dose) samples, but BEI treatment apparently eliminated the reactive endotoxin in the LAL endotoxin assay.

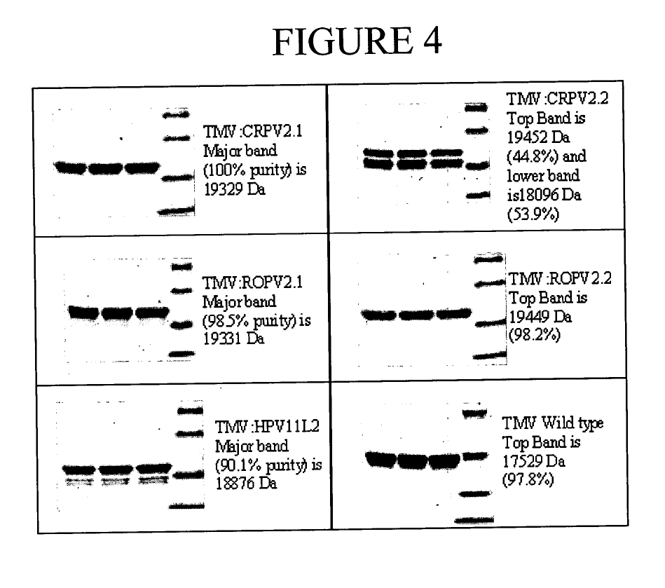

Two microgram samples of final fill vaccines, both untreated and BEI treated were run in triplicate on 10-14% Tris-HCl SDS-PAGE gels, and stained with Coomassie brilliant blue. FIG. 4 shows the results of these analyses.

All vaccines, with the exception of TMV:CRPV2.2, contain >90% fully intact recombinant coat protein. MALDI-TOF analysis confirms that, in all cases, the upper band in the virus preparations contains the full B-cell epitope amino acid sequence as predicted from the DNA sequence of the clone. About half of the TMV:CRPV 2.2 vaccine is fully intact. MALDI-TOF analysis of tryptic fragments of the TMV:CRPV2.2 product indicate that the first 10 amino acids of the 14 amino acid epitope are present in the smaller (18 096 and 17985) bands.

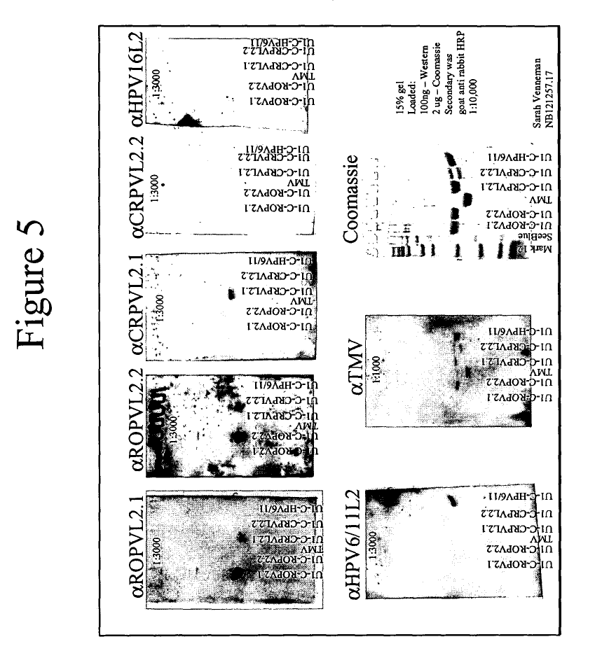

Membranes with TMV: papillomavirus vaccine antigens were probed with rabbit antisera specific for rabbit or human papillomaviruses by Western blot analysis. The results are shown in FIG. 5: there is some cross-reactivity between ROPV2.1 and CRPV2.1. The CRPVL2.2 sera reacts only weakly to the vaccine antigen, but all other sera react specifically with the vaccines.

Preliminary immunogenicity testing of the papillomavirus:TMV epitope fusions was performed to ensure that appropriate antibody responses could be induced by immunization of animals with the vaccines, and to determine what, if any, effect BEI-inactivation of the TMV virions would have on the immunogenicity of the recombinant viruses. Four to five week old, female BALB/c mice were used to assay immunogenicity of the vaccines, and to compare the immunogenicity of BEI-inactivation TMV preparations with untreated controls. In addition, we immunized a small number of female guinea pigs to confirm that the vaccines were immunogenic in more than one species of animal, and also to generate antisera that could be used in in vitro virus neutralization studies (to be performed at Pennsylvania State University).

Four animals per group received a dose of 10 μg of the TMV vaccine product, administered subcutaneously. Vaccines were administered every second week, and a total of four vaccines were given. All six BEI-inactivated vaccines were administered, and untreated (non-BEI inactivated) versions of the TMV, TMV:CRPV2.1 and TMV:HPV11L2 vaccines were given to serve as controls for the BEI-inactivated vaccines. One further group received a mixed vaccine series containing 5 μg each of TMV:CRPV2.1 and TMV:CRPV2.2 to establish whether an immune response to two different epitopes could be induced with a mixed vaccine. No PBS control was used, as each vaccine could serve as a control for the others. Animals were bled from the tail vein, after mild hyperthermia, nine days after vaccines 2, 3 and 4. ELISA, using peptide-conjugated bovine serum albumin as the capture antigen, determined antibody titers. Rabbit polyclonal sera specific for the peptide epitopes were provided by Neil Christensen, and served as positive controls, and tittering standards on ELISA plates. The rabbit sera used as positive control were: HPV1/11 NC25 C000840; CRPVL2.1 B0229; CRPVL2.2 B0225; ROPVL2.1 B0219 and ROPVL2.2 B0220. For comparison of ELISA titers with the rabbit sera, a dilution of the rabbit sera was chosen, and arbitrarily set to 1. The mouse antibody titers were expressed as a unit of the rabbit sera. The subclasses of antibodies of the IgG isotype were measured with secondary antibodies specific for mouse IgG1 or IgG2.

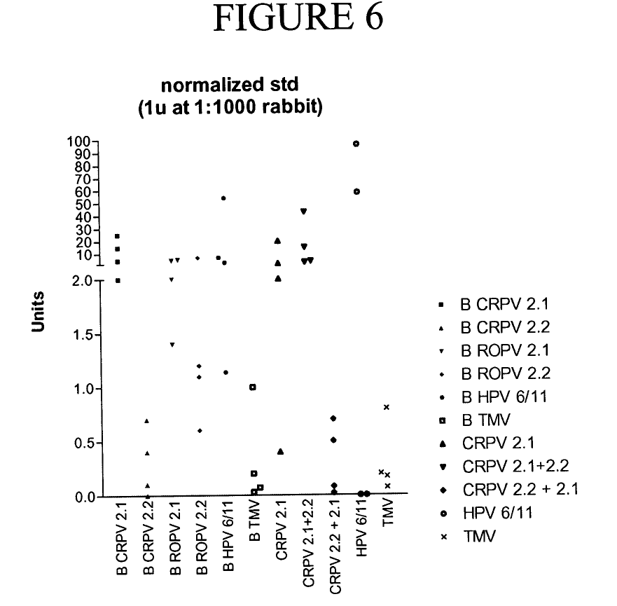

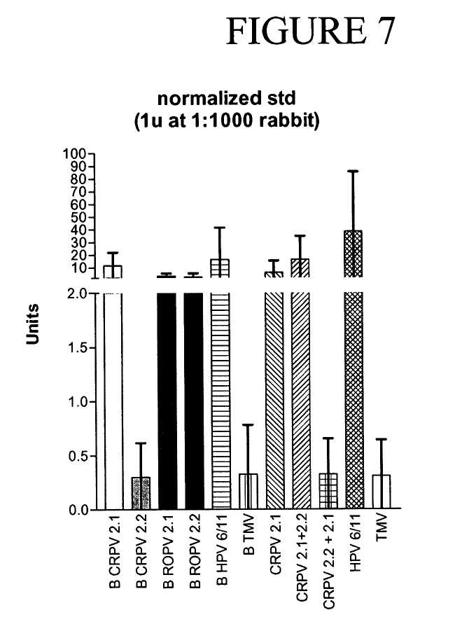

FIG. 6 shows a scatter plot of antibody responses of all vaccinated animals to the peptide antigen; FIG. 7 shows the same data in bar graph format, with error bars indicating 95% confidence intervals. The X axis standard is normalized to the various rabbit positive control sera, where 1 unit is the OD obtained for a 1:1000 dilution. This gives some indication of the range of responses seen in each group, relative to the positive control sera. The responses to different antigens are obviously impossible to compare, since the antibody titer in the positive control sera are not standardized to each other. However, the data show the variability we observed in immune response, and the magnitude of the response relative to the rabbit control sera supplied by Neil Christensen (Pennsylvania State University, Hershey Pa), at a 1:1000 dilution. On the Y-axis, the different experimental groups are listed, with the prefix B-indicating BEI-inactivated samples, and no prefix indicating untreated samples. Peptide-BSA conjugates were used as coating antigens, except for the TMV samples, where wild type TMV was used. For the mixed vaccine (CRPV2.1+CRPV2.2), CRPV 2.1 peptide was used as the coating antigen when the label indicates CRPV2.1 first; and vice-versa.

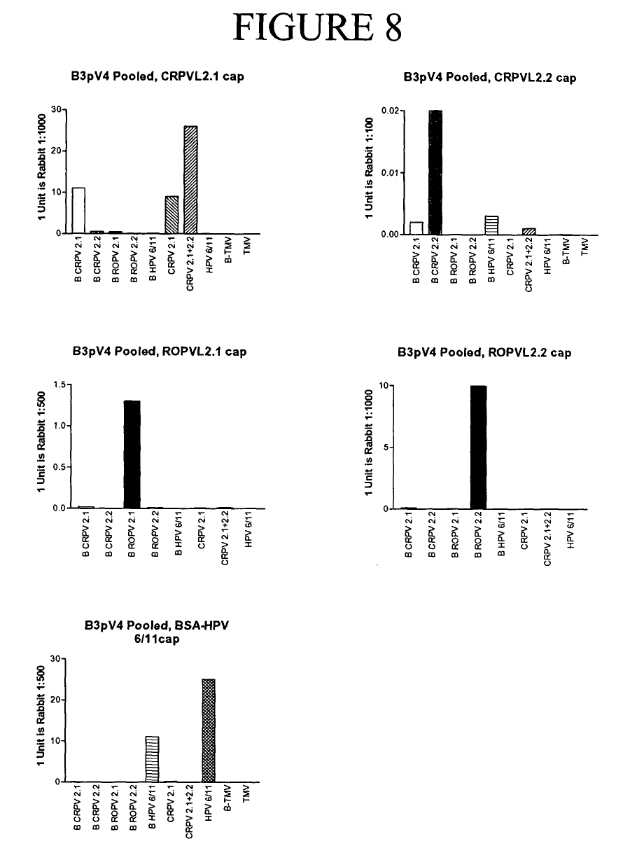

FIG. 8 shows an analysis of the antigen-specificity of sera from vaccinated animals. Pooled sera were reacted with plates carrying all of the different peptide antigens. The antibodies appear very specific, in all cases, with no, or very little cross-reactivity between antigens.

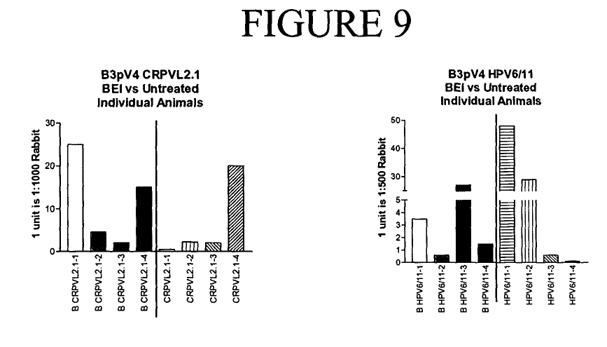

The effect of BEI inactivation of TMV peptide vaccines, with untreated samples was compared. The data depicted in FIG. 9 show that the immune response of animals vaccinated with BEI-inactivated TMV:CRPV2.1 and untreated TMV:CRPV2.1 was qualitatively similar. Likewise, animals vaccinated with BEI-inactivated TMV:HPV6/11L2 and the untreated version reacted similarly. In FIG. 8 we show a comparison of the IgG subtype profile in pooled sera from animals vaccinated with all of the vaccines. Note that BEI inactivation of the CRPV2.1 and HPV6/11 vaccines seemed to have no major effect on the quality of the immune response, as measured by IgG subtype.

An immunogenicity study in guinea pigs was performed in addition to the mouse study described above. A total of six animals were used in this study; two animals each received the TMV:CRPV2.1; TMV:HPV6/11; and mixed TMV:CRPV2.1 plus TMV:CRPV2.2 vaccines. The dose of vaccine was 100 μg, administered every second week. A total of four vaccines were given. Each dose was administered subcutaneously, at four locations on the animal's back. Animals were bled one week post vaccine 3 (bleed 1) and one week post vaccine 4 (bleed 2). A terminal bleed was collected nine days after vaccine 4.

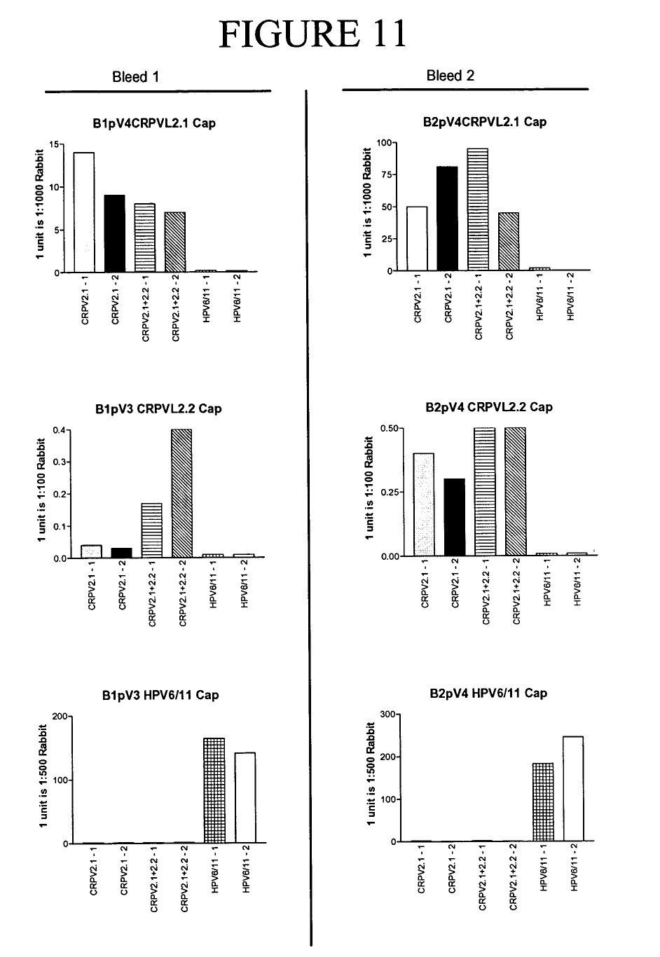

The ELISAs were performed in the same way as for the mouse study. FIG. 11 shows the antibody titer obtained for each individual animal after vaccine 3 (left) and after vaccine 4 (right). We note that, as for the mouse vaccinations, the anti CRPV2.2 peptide response was very low, and only marginally above background. It is possible that in this vaccine, which contained more than 50% cleavage, a new epitope comprising the part of the TMV coat protein and part of the first 10 amino acids of the CRPV2.2 peptide is recognized and is dominant over the authentic CRPV2.2 epitope. These results argue for elimination of the TMV:ROPV2.2 vaccine from subsequent studies.

The titer of the CRPV2.1 and HPV6/11 peptide antibodies was significantly higher in the Guinea pig sera in comparison with the BALB/c mouse sera. In all cases, both guinea pigs responded well to the vaccine; apparently well within a Log of the rabbit titer. It is worthwhile noting that the mice received 1/10 of the vaccine dose that the guinea pigs received, and that the higher dose could have had some positive effect on the immune response observed in the guinea pigs.

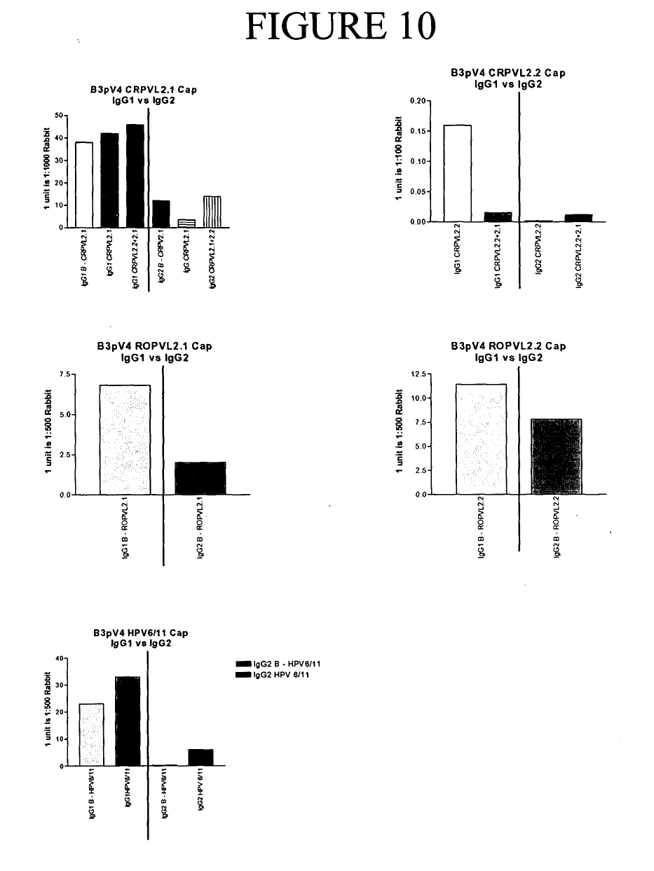

The IgG subtype analyses presented in FIG. 10 show that the guinea pigs responded similarly to the mice to the vaccines: with a balanced, but apparently Th2-dominant response.

Bleeds 1 and 2 and terminal bleeds from all the guinea pigs, and terminal bleeds from highest mouse responder in each group are available for CRPV and HPV6 or HPV 11 neutralization assays.

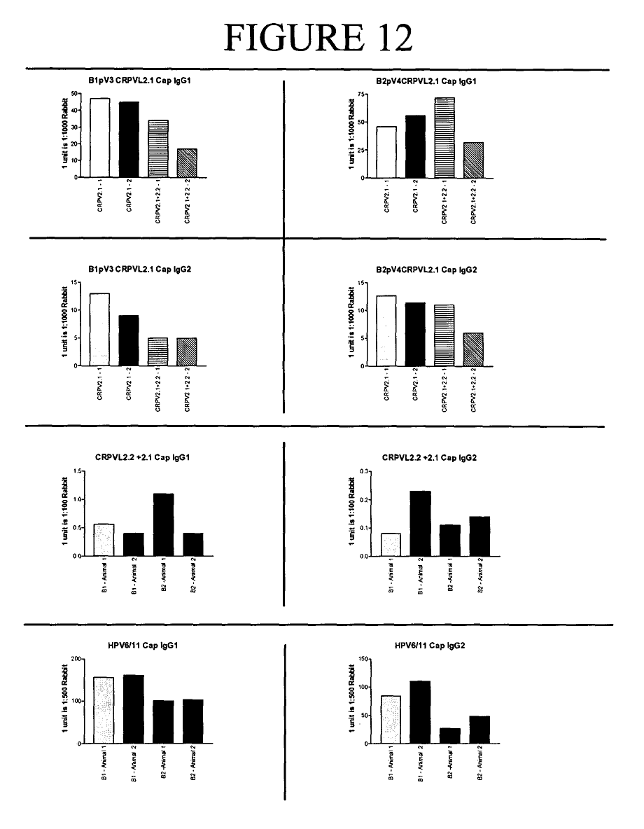

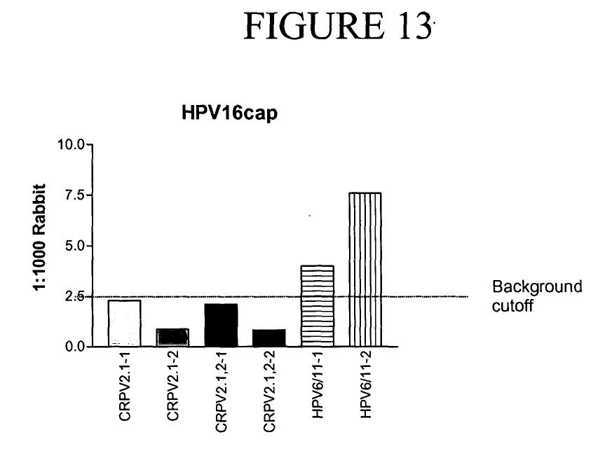

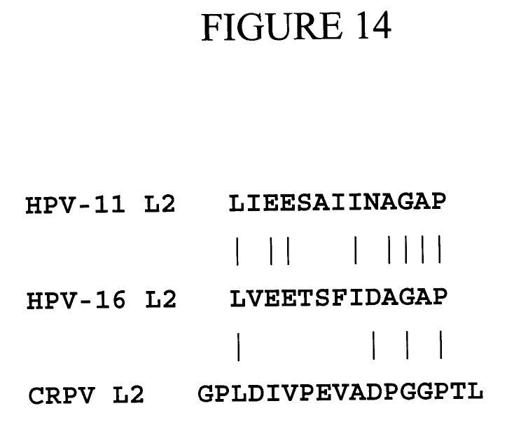

We investigated whether sera from animals immunized with TMV virions displaying the HPV6/11 L2 epitope (LIEESAIINAGAP) could recognize the HPV 16 L2 epitope sequence LVEETSFIDAGAP, conjugated to BSA, and used as a coating antigen on ELISA plates. FIG. 12 shows that, indeed sera from the guinea pigs immunized with the TMV virions displaying the HPV6/11 L2 epitope (LIEESAIINAGAP) specifically recognized the heterologous HPV 16 L2 peptide sequence (LVEETSFIDAGAP). These data are shown in FIG. 13, and indicate that immunization with TMV virions displaying the HPV6/11 L2 peptide sequence may function as prophylactic vaccines that can induce broadly neutralizing papillomavirus L2-specific antibodies. The homology between the HPV6/11 L2 epitope, the HPV-16 L2 epitope and the CRPV2.1 epitope are shown in FIG. 14.

### EXAMPLE 6

### Carrier Rotation to Improve Immunological Responses to Peptide-Based Vaccines

Virus like particle (VLP)-based vaccines can carry specific antigens and to be particularly effective in inducing humoral, and sometimes, cellular immune responses. It is now well established that peptides are most efficiently presented to the mammalian immune system in a highly ordered, repetitive, quasicrystalline array as provided by a VLP structure (Bachmann et al., 1993; Savelyeva et al., 2001). By their structure, VLPs are capable of stimulating proliferation of dendritic cells and other antigen presenting cells resulting in strong immunological responses thus producing protective immunity and even breaking tolerance for self-antigens (Savelyeva et al., 2001; Fitchen et al., 1995). This structural presentation of antigens appears to be critical for induction of strong Th1 or Th2 responses (including antigen specific CTL responses and long-lived B cell memory) and cannot be replicated by soluble proteins or randomly conjugated carriers, such as KLH (Storni et al., 2002; Nicholas et al., 2002). These results have led to a great deal of interest in VLP epitope display systems for induction of pathogen-specific antibodies for protection against infectious disease, as well as for induction of peptide-specific CTL responses in immunotherapy of cancer and chronic infectious diseases. There are many candidates for VLP technologies, but hepatitis B core antigen (HBcAg) and papillomaviruses represent well-established methodologies for recombinant production of VLP-epitope display. HBcAg VLPs are produced recombinantly in E. coli systems and are effective tools for VLP display (Bachman and Kopf, 2002). Purification of endotoxin-free structures is a challenge from such systems. In addition, the rate of successful expression of epitopes genetically fused to these structures is highly variable. Some groups have addressed this issue by resorting to in-vitro methods for conjugating synthetic peptides to VLPs, but these methodologies do not necessarily replicate the structural advantages of native VLP structures, and are technically challenging and expensive to perform, especially at large scale. Preexisting immunity can blunt immune responses to VLP carriers. Da Silva et al., (2001) reported that preexisting neutralizing antibodies to human papillomavirus L1 virus like particles limit the effectiveness of vaccines that use this carrier for subsequent inoculations. It should be noted that significant preexisting immunity exists in the human population for these viruses (up to 20% by some estimate).

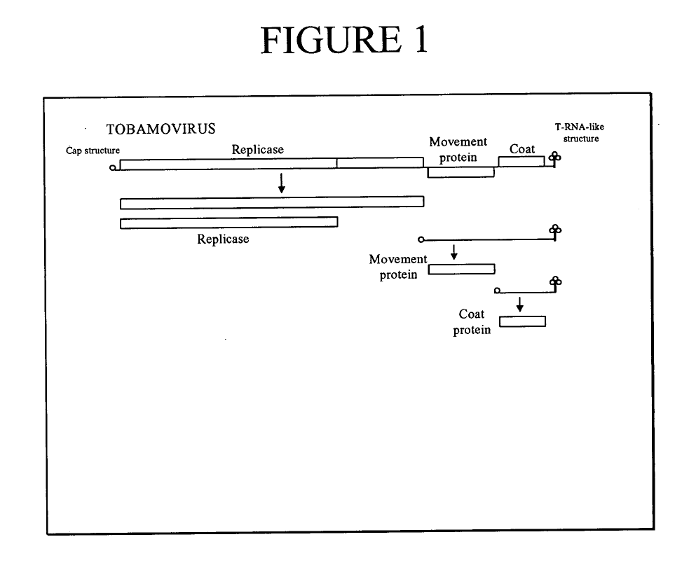

The tobamovirus family, including TMV, offers the tools for building a robust epitope display vaccine platform. Each of the 13 tobamovirus species encodes a coat protein with similar structural folding (Stubbs, 1999). Each coat protein exhibits surface exposed N and C termini (extreme end and upstream of terminal GPAT motif) and a single surface-exposed loop (“60's loop) that have been shown experimentally to tolerate insertion of peptide sequences (FIG. 1; see references within 1). Although conserved in overall structure, TMV strains U1, U5, cucumber green mild mottle virus (CGMMV), and ribgrass mosaic virus (RMV) are all immunologically distinct, while TMV U1 and ToMV are immunologically similar (Jaegle and Van Regenmortel, 1985; Gibbs 1999; 1997). Studies of mammalian immune responses to tobamoviruses pioneered our understanding of host responses to virus structures and have continued for over 60 years (Van Regenmortel, 1999). Extensive studies by phytopathologists determined that mammals immunized with tobamoviruses produce antibodies with little cross-reactivity with other tobamovirus coat proteins. This structural conservation, coupled with immunologic distinctness, provides a unique opportunity for deriving a platform of vaccine protein scaffolds that share similar biochemical and purification properties.

Display of peptides on TMV VLPs may be used for the induction of neutralizing responses to biodefense related pathogens was illustrated by VLP vaccine candidates generated against the filovirus pathogen Ebola. Additional biodefense related epitopes have been identified for bacterial and viral pathogens and include the Rift Valley Fever neutralization epitope KGTMDSGQTKREL bound by protective Mab 4D4 (Keegan and Collett, 1986; London et al., 1992). We have also made the TMV virions displaying peptides specifically binding neutralizing antibodies against the Ebola virus (Wilson et al., 2000). The minimal consensus sequence, underlined, represents the common sequence found on two adjacent overlapping peptides that were bound by the neutralizing MAb:

1. Ebola glycoprotein 389-405 HNTPVYKLDISEATQVE

2. Ebola glycoprotein 401-417 ATQVEQHHRRTDNDSTA

3. Ebola glycoprotein 477-493 GKLGLITNTIAGVAGLI

Fusion proteins of these minimal consensus peptides were generated at the N-terminal, 60's loop, and near the C-terminal of the TMV U1 coat protein using the general techniques above. The solubility of peptides fused to the coat proteins extracted from N. benthamiana plants inoculated with infectious transcripts is shown in Table 6 and FIG. 15. The virions that remain soluble in aqueous solutions differ in terms of the absolute yield of recombinant virus recovered from infected tissues, and the optimal buffer extraction conditions necessary for extraction. For example, the epitope GP1-481 fused to N-terminal of coat protein has a slightly lower yield compared to the same epitope fused near the C-terminus of the TMV U1 coat protein. The majority of the virion with an N-terminal GP1-481 fusion is soluble in TRIS-C1 buffer (pH 7.5), whereas the virion carrying the same fusion near the C-terminus was soluble at either pH 5.0 or 7.5. As expected, the negative control samples did not have any SDS-PAGE band near the expected size of the coat protein fusions. The integrity of the fusions was further confirmed by MALDI-TOF mass spectroscopy (Table 6). Viral constructs with the epitope fused in the 60's loop caused necrotic lesions on N. benthamiana plants and often resulted in insoluble recombinant coat protein. Approaches to overcome this problem include testing these constructs in other Nicotiana species or changing the amino acid sequences surrounding the epitope to restore the native charge of (−3) on the TMV U1 coat protein. From these data, it is clear that peptide epitopes bound by antibodies capable of neutralizing Ebola virus and protecting mice from infection were readily displayed on the surface of TMV virions.

The cloning vectors for fusing peptides to various tobamovirus coat proteins were constructed using unique restriction endonuclease sites, PCR-based genetic fusions and insertion cloning procedures. For example, for displaying epitopes on the U1 coat protein, vectors possess unique NcoI and NgoMIV restriction sites at four locations, N-terminal, C-terminal, C-terminal upstream of the GPATmotif, and within the surface exposed loop region. These linearized sites can readily accept any hybridized oligonucleotides (coding for epitopes) with the same overhangs. We will use the same strategy to prepare cloning vectors for the other three coat proteins. Recombinant virus clones were transcribed and capped in-vitro, and the infectious transcripts were inoculated onto plants: N. benthamiana or N. excelsiana. Infections of plants were scored visually between 5 and 10 days post inoculation.

A low pH buffer (50 mM sodium acetate, 5 mM EDTA, pH 5.0) was very useful for initial extraction of virus coat protein fusions since many host proteins are insoluble at this pH and so coat protein bands are easily visible in extracts run in SDS-PAGE gels and stained with Coomassie Brilliant Blue. However, several coat fusions were not soluble under these conditions. Some of these were selectively solubilized from insoluble plant material by resuspension of the material in 50 mM Tris-HCl pH 7.5 buffer followed by centrifugation to remove insoluble materials. The virus was purified by differential centrifugation followed by precipitation of virus by treatment of supernatants with 4% polyethylene glycol in the presence of 0.7M NaCl. Accurate sizing of coat protein subunits was possible by MALDI-TOF mass spectrometry, and that this methodology was very useful for verification that fusion proteins were intact, and not proteolytically cleaved. When further verification of protein identity was required, the band can be excised from gels and subject to digestion with trypsin followed by MALDI-TOF for verification that the predicted tryptic digest matches the observed pattern of ion masses in the MALDI-TOF spectrum. Recombinant fusions that are soluble in either pH 5 or pH 7.5, can be readily manufactured for vaccine investigations.

Peptide display vaccines applied with a single carrier can induce a response primarily to the carrier protein, rather than effectively boosting immune responses to the peptide antigen. To discourage such carrier-specific boosting responses, a carrier rotation approach to vaccines was used. In this case, the peptide immunogen, such as Ebola neutralizing peptide GP1-393 (VYKLDISEA), was fused to the surface of the coat protein of TMV U1 and TMGMV or RMV coat protein. The initial immunization was given with the TMV U1-peptide vaccine and the boosting immunization will be given 2-4 weeks later using the TMGMV or RMV fusion. In this manner, the immune system of the immunized individual sees only one consistent linear epitope, and that is for the peptide immunogen. This enhances the level of immune response and the specificity of the immune response over that available for a vaccine using a single carrier in repeated immunizations. The principle is useful for any peptide or protein antigen which is presented with a non-specific antigen. The booster effect of multiple vaccinations is then directed only to the specific peptide immunogen, not to the carrier molecule or portion or the carrier molecule.

This concept was extended to a multi-peptide immunogen vaccine. In this case, a set of peptide immunogens was employed in a vaccine to induce a wider anti-pathogen response against a single organism (e.g. Ebola: peptides GP1-393, 405, 481). In contrast, a set of peptide immunogens to different organisms can be applied in a single vaccine to induce an effective immune response against more than one organism simultaneously (e.g. Ebola, GP1-393 and RVFV 4D4 peptide). Each is fused to the surface of the coat protein of TMV U1 and TMGMV or RMV coat protein. The initial immunization is given with the TMV U1-peptide vaccines and the boosting immunization will be given 2-4 weeks later using the TMGMV or RMV fusions. In this manner, the immune system of the immunized individual sees only the two (or more) consistent linear epitopes that are for the multi-pathogen peptide immunogens. This approach enhances the level of immune response and the specificity of the immune response over that available for a vaccine using a single carrier in repeated immunizations.

In either situation or carrier rotation, the epitope peptide may be fused to the carrier antigen or it may be mixed therewith to present or enhance the immune response. Plural epitope peptides may be bound to the same or different carrier antigens simultaneously. In situations where many immunizations to the same peptide epitope are desired, such as for allergy treatments, this method is particularly useful. Also, when one does not know which peptide epitope is best to use for immunization, to produce neutralizing antibodies for example, one may prepare many vaccine preparations without concern for the carrier antigen becoming immunodominant.

A murine model for Ebola filovirus is an example of the test systems that may be used to for such a rotating carrier approach. Murine test hosts were of the Balb C or C57B1/6 mouse strains. Mice were immunized with VLP peptide vaccines (a dose range (2 and 10 mcg) fused to TMV U1 (first immunization), TMGMV or RMV (second and/or third immunization). Peptides were chosen from the group (peptides GP1-393, 405, 481) and PBS buffer was used a negative control. Mice were immunized at two week intervals. Sera from each mouse, pre-immune and two weeks following each immunization, were screened against each the VLP vaccines displaying the cognate peptide on the surface of either TMV U1, TMGMV or RMV by ELISA. MAbs that recognize different Ebola antigens (6D8-1-2, 13F6-1-2 and 12B5-1-1, kindly provided by Dr. Mary Kate Hart, US AMRIID) recognized the cognate linear neutralizing epitopes on the different carriers with peptides. ELISA assays were completed as described (40). Briefly, Nunc Maxisorp 96 well plates were coated overnight with 5 μg/ml of target antigen in carbonate buffer. Targets included cognate peptide conjugated to BSA, TMV-Ebola peptide fusion, TMV-RVFV peptide fusion, and TMV. Plates are washed, blocked, and incubated with a 1:3 serial dilution of sera from immunized or control mice at a starting dilution of 1:10. Plates were then washed, and incubated with an anti-mouse-HRP conjugate. Following secondary incubation, plates were washed, and developed by standard procedure, and read on a Molecular Devices Gemini plate reader at 405 nm. The level of bound antibodies were determined by comparing to the known amount of neutralizing MAb.

Sera derived from immunized mice were tested for their ability of these immune sera to inhibit or alter Ebola virus plaque formation. Sera showing the most robust anti-peptide immune responses were used. Neutralization assays were carried out as described in Wilson et al., (2). Briefly, fourfold serial dilution of sera was mixed with 100 pfu of murine-adapted Ebola Zaire at 37° C. for 1 hour in the presence or absence of 5% guinea pig complement (Accurate Scientific) and used to infect Vero E6 cells. Cells were overlaid with agarose and a second overlay with 5% neutral red added 6 days later. Plaques were counted on the 7th day. Neutralization titers were determined to be the last dilution of the sera that reduced the number of plaques by 80% compared with control wells (sera from PBS or RVFV peptide immunized mice).

The Ebola peptide immunogens fused to tobamovirus VLP structures can be tested for efficacy in an Ebola challenge model. Ten mice per treatment will be evaluated in each of two experiments. C57BL/6 mice will be vaccinated at two doses at 4 week intervals and challenged intraperitoneally with 1000 pfu of mouse-adapted Ebola Zaire virus (2; 43) one month after the final immunization. Mice will be observed daily for signs of illness for 28 days after challenge.

### EXAMPLE 7

### Carrier Rotation to Improve Immunological Responses to HIV Vaccines

Ideally, a vaccine designed to protect against infection with human immunodeficiency type 1 (HIV-1) will induce sterilizing immunity against a broad range of virus variants. However, generation of broadly-neutralizing antibodies (Nabs) by vaccination, let alone natural infection, has proven nearly impossible thus far. There have been some notable advances in development of vaccine regimens that are able to generate significant levels of protection against development of AIDS in non-human primate models (reviewed in McMichael et al., 2002; Letvin et al., 2002; Robinson 2002; Letvin 2002). These vaccines allow animals to control viral challenge by strong priming of virus-specific CD8⁺ T-cells (cytotoxic T cells, CTLs). However, a CTL response alone cannot prevent infection, and mechanisms to induce Nabs that will neutralize a wide range of isolates remains a vital goal, especially in light of the fact that viral escape from vaccine-induced CTL control can sometimes occur (Barouch et al., 2002). The Env spikes on the surface of the HIV-1 virion are the primary target for antibody-mediated neutralization. However, the Env proteins of HIV-1 are poorly antigenic, and generation of Nabs is difficult to achieve, probably because functionally important domains of the proteins are obscured by protein folding and carboydrate chains. Nevertheless, many infected people do mount a Nab response that is generally highly specific to the autologous virus, and not cross-neutralizing. This is not surprising given the phenomenal sequence and structural variation that is present in the Env proteins. However, a rare subset of infected individuals do produce broadly neutralizing Abs, which gives hope that induction of sterilizing immunity is possible.

The envelope proteins of T-cell line-adapted (TCLA) strains of HIV-1 elicit Nabs that mostly target linear epitopes in the third variable cysteine loop (V3 loop) of gp120, a region that is involved in co-receptor binding and hence vital for virus entry. Subtype C isolates of HIV-1, which infect more people worldwide than any other subtype, have relatively low level of sequence variation in the V3 loop (Engelbrecht et al., 2001; Bures et al., 2002). However, neutralization of subtype C virus by V3 loop Abs is not extremely efficient in vitro, perhaps reflecting poor immunogenicity of epitopes in this region (Bures et al., 2002). There is concern that the V3 loop may be hidden in the native gp120 structure and not accessible to the immune system, and therefore that generation of V3-specific Nabs will be difficult with gp120 subunit vaccines. However, the V3 loop is vital for viral entry, and so significant levels of V3 loop-targeted Nabs should help prevent transmission of HIV-1.

To date, six human monoclonal antibodies (Mabs) have been described that are capable of neutralizing a broad spectrum of HIV-1 variants in vitro. Three of these (IgGb12; 2G12 and 2F5) were described several years ago, and lend insight into the domains of the Env proteins that are important in viral entry, and thus for vaccine design. Monoclonal antibody “b 12” recognizes a conformational epitope in the CD4 binding site of gp120; 2G12 recognizes a discontinuous epitope in the C2-V4 region of gp120 that includes N-glcyosylation sites, and 2F5 maps to a linear epitope (ELDKWA) in the membrane-proximal ectodomain of gp41 (D'Souza et al., 1997). Recently, two broadly neutralizing monoclonal antibodies 4E10 and Z13 were shown to recognize a continuous epitope with core sequence NWFDIT, just C-terminal to the 2F5 recognition sequence (Stiegler et al., 2001; Zwick et al., 2001). This strongly indicates that the membrane proximal region of gp41 plays a critical role in virus entry. Another recently described monoclonal Fab was selected for binding to gp 120-CD4-CCR5 complexes, and also displays a broad neutralization phenotype (Moulard et al., 2002).

Passive transfer studies have shown that neutralizing Mabs are able to confer concentration-dependent sterilizing immunity to virus challenge by intravenous, oral and vaginal routes in Rhesus macaques. It is encouraging that the mAbs tested display significant synergy in their neutralization activity: this will reduce the minimum antibody concentration that is required for effective neutralization (reviewed in Mascola, 2002; Xu et al., 2002). A recent publication (Lewis et al., 2002) demonstrates that MAb neutralizing activity can also be generated in vivo: in mice that expressed the gene for b12 from a recombinant adeno-associated virus vector. These studies on neutralizing Mabs have helped to demonstrate that we should be able to achieve significant levels of protection against HIV-1 infection and reduced rates of transmission of virus, if a way is found to induce robust production of Nabs in vaccinated animals and is incorporated into a vaccine regimen that includes strong priming of a CTL response.

In the light of the disappointing performance of whole Env-based vaccines, and the problems associated with poor immunogenicity of Env subunit vaccines, several studies have focused on the use of immunogens based on domains of Env proteins that are presumed targets for Abs. Data presented by Letvin et al. (2001), that showed that antibodies induced against the V3 loop could provide partial protection against challenge with primary isolate-like SHIV-89.6 in Rhesus macaques. Efforts at generation of neutralizing antibodies with immunogens containing the core linear epitope recognized by the 2F5 antibody have been generally disappointing, with only non-neutralizing antibodies being produced (Ferko et al., 1998; Echart et al., 1996). However, there is one notable exception: recently, Marusic et al. (2001) showed that virus-like particles of the flexuous plant virus potato virus X (PVX) dispaying the 2F5 ELDKWA epitope could induce high levels of HIV-1 specific IgG and IgA in mice immunized with the recombinant virus-like particles (VLPs). This immunogen was able to induce production of human HIV-1 specific neutralizing antibodies (measured by in vitro inhibition of syncytium formation) in severe combined immunodeficient mice reconstituted with human periferal blood lymphocytes (hu-PBL-SCID) that had been immunized with human dendritic cells (DCs) pulsed with the PVX-2F5 VLPs. These authors speculate that presentation of the ELDKWAS sequence in a highly repetitive fashion on the surface of the PVX virion rendered the sequence highly immunogenic, and thus were able to generate Nabs.

Until the recent discovery of the 4E10/Z3 human Mab, 2F5 was the only human Mab that appeared to recognize a linear epitope, and so peptides that could mimic the neutralizing epitope of b12 and 2G12 were not available for testing as potential immunogens. However, a linear peptide mimotope of the b12 epitope has recently been discovered using phage peptide display technology (Zwick et al., 2001). This peptide (B2.1) appears to bind best to b12 when presented as a disulphide-linked homodimer on the surface of the phage. This phage particle is being optimized for use as an immunogen. Scala et al. (1999) selected epitopes from libraries of peptides displayed on the surface of filamentous phage particles with sera from HIV⁺ patients, both from long term infected non-progressor donors and from donors who had progressed to AIDS illness. Five epitopes, presumed to be mimotopes of Env-specific neutralizing epitopes, were able to induce production of antibodies that neutralized TCLA HIV-1 strains IIIB and NL4-3, as well as the primary isolate AD8, but this less strongly than the TCLA strains (Scala et al., 1999). Subsequently, these authors showed that sera from individuals infected with all group M HIV-1 subgroups were able to recognize the phage-displayed mimotopes (Chen et al., 2001). Rhesus monkeys were immunized with phage particles displaying the five epitopes that had shown potentially protective immune responses in mice, and challenged with pathogenic SHIV-89.6PD. While the immunized animals were not protected from SHIV infection, there was evidence of significant control of the challenge virus and the monkeys were protected from progression to AIDS. These results show similar levels of control to vaccines designed to generate virus-specific CTLs and infer that the antibody response was able to control viremia in the challenged animals. A recent publication (He et al., 2002) described successful isolation of a number of human Nabs from XenoMouse immunized with gp120 derived from a primary Subtype B isolate (SF162). The authors noted potent neutralizing activity against the autologous virus isolate, and reactivity against both R5 and X4 isolates in Subtype B. The Nabs mapped to novel epitopes in domains known to possess neutralizing epitopes: V2-, V3- and CD4-binding domains of gp120, as well as in the C-terminal region of the V1 loop. Apparently, several Nabs recognize linear epitopes that now warrant further investigation as peptide immunogens.

Some non-structural HIV-1 proteins, particularly Tat and Vpr, are found in the serum of infected individuals, and exert biological function, resulting in immunodeficiency and disease. The Tat protein is required for HIV-1 replication and pathogenesis. It is produced early in the viral life cycle. In the nucleus of the infected cell, it interacts with host factors and the TAR region of the viral RNA to enhance transcript elongation and to increase viral gene expression (Jeang et al., 1999). Tat also is also found extracellularly, where it has distinct functions that may indirectly promote virus replication and disease, either through receptor mediated signal transduction or after internalization and transport to the nucleus. Tat suppresses mitogen-, alloantigen- and antigen-induced lymphocyte proliferation in vitro by stimulating suppressive levels of alpha interferon and by inducing apoptosis in activated lymphocytes. In vivo, it is thought that Tat may alter immunity by upregulating IL-10 and reducing IL-12 production, or through its ability to increase chemokine receptor expression (Gallo et al., 2002; Tikhonov et al., 2003). Antibody production against Tat has, in some cases, correlated with delayed progression to AIDS in HIV-1 infected people (Gallo et al., 2002). Recently, Agwale et al. (2002) showed that antibodies induced in mice against a Tat protein subunit vaccine could negate the immune suppression activities of Tat in vivo. Subsequently, Tikhonov et al. (2003) identified linear epitopes on Tat that were reactive with Tat-neutralizing antibodies produced in vaccinated Rhesus macaques. From these data it is clear that antibodies that target the N-terminus, an internal basic domain, and the cell-binding domain of Tat (containing the integrin-binding motif “RGD”) can neutralize the extracellular version of Tat, and reduce the negative impact of Tat on the immune system. These linear epitopes are thus interesting targets for both prophylactic and therapeutic vaccines against HIV-1 and AIDS.

As in the examples above, peptide epitopes were prepared in TMV coat proteins and produced as above. In Table 7, a list of peptides that have been displayed on the surface of TMV U1 and/or U5 virions is displayed. The expression, extraction and solubility data for these recombinant viruses is summarized in Table 8.

### EXAMPLE 8

### Veterinary Parvovirus Vaccines

Parvoviruses that are associated with enteric disease in domestic cats, dogs, mink and pigs are closely related antigenically, with different isolates diverging less than 2% in the sequence of the viral structural proteins. Vaccination with killed or live-attenuated parvovirus protects animals against infection by Feline panleukopenia virus (FPV), canine parvovirus (CPV), mink enteritis virus (MEV) and porcine parvovirus (PPV). However, maternal antibodies neutralize the vaccine, making it ineffective in animals that have not been weaned. Subunit vaccines might overcome this limitation, and provide useful alternatives to conventional vaccines.

The N-terminus of FPV, CPV and PPV VP2 contains a major neutralizing determinant for the virus; this is a linear epitope, present in the first 23 amino acids of the protein. Neutralizing antibodies may be induced in animals immunized with peptides derived from the first 23 amino acids of VP2 (Casal et al., 1995; Langeveld et al., 2001). The sequence of the N-terminus of VP2 follows: MSDGAVQPDGGQPAVRNERATGS

We designed a synthetic DNA sequences which would encode various portions of the N-terminal VP2 sequence. The synthetic DNA was synthesized in complementary oligonucleotides, and inserted into the coat protein of TMV U1 and TMV U5, as depicted in FIG. 2. These sequences of the peptides were denoted Parvol; Parvo2; and Parvo3. The amino acid sequences of these peptides are as follows:

| | | |
|---|---|---|
| Parvo1: | MSDGAVQPDGGQPAVRNERAT | (21 amino acids) |
| Parvo2: | MSDGAVQPDGGQPAVRNERA | (20 amino acids) |
| Parvo3: | VQPDGGQPAVRNERAT | (16 amino acids) |

These epitope peptide vaccines are then used as in the examples above It will be understood that various modifications may be made to the embodiments disclosed herein. Therefore, the above description should not be construed as limiting, but merely as exemplifications of preferred embodiments. Those skilled in the art will envision other modifications within the scope and spirit of the claims appended hereto.

All patents and references cited herein are explicitly incorporated by reference in their entirety.

### References

Agwale S M, Shata M T, Reitz M S Jr, Kalyanaraman V S, Gallo R C, Popovic M, Hone D M. (2002) A Tat subunit vaccine confers protective immunity against the immune-modulating activity of the human immunodeficiency virus type-1 Tat protein in mice. Proc. Natl. Acad. Sci. USA 99:10037-41.

Alani R M, Munger K (1998) Human papillomaviruses and associated malignancies. Journal of Clinical Oncology 16:330-337.

Bachmann M F, Kopf M. (2002) Balancing protective immunity and immunopathology. Curr Opin Immunol. 14:413-9.

Bachmann M F, Rohrer U H, Kundig T M, Burki K, Hengartner H, Zinkernagel R M. (1993) The influence of antigen organization on B cell responsiveness. Science. 262:1448-51.

Balaram P, Nalinakumari K R, Abraham E, Balan A, Hareendran N K, Bernard H U, Chan S Y. (1995) Human papillomaviruses in 91 oral cancers from Indian betel quid chewers—high prevalence and multiplicity of infections. Int J Cancer. 61:450-4.

Barouch D H, Kunstman J, Kuroda M J, Schmitz J E, Santra S, Peyerl F W, Krivulka G R, Beaudry K, Lifton M A, Gorgone D A, Montefiori D C, Lewis M G, Wolinsky S M, Letvin N L. (2002) Eventual AIDS vaccine failure in a rhesus monkey by viral escape from cytotoxic T lymphocytes. Nature. 415:335-9.

Borio L, Inglesby T, Peters C J, Schmaljohn A L, Hughes J M, Jahrling PB<Ksiazek T, Johnson K M, Neyerhoff A, O'Toole T, Ascher M S, Bartlett J, Bremen J G, Eitzen E M, Hamburg M, Hauer J, Henderson D A, Johnson R T, Kwik G, Layton M, Lillibridge S, Nabel G J, Osterholm M T, Perl T M, Russell P, Tonat K, for the Working Group on Civilian Biodefense. 2002. Hemorrhagic fever viruses as biological weapons. JAMA 287:2391-2405.

Breitburd F, Kirnbauer R, Hubbert N L, Nonnenmacher B, Trin-Dinh-Desmarquet C, Orth G, Schiller J T, Lowy D R. (1995) Immunization with viruslike particles from cottontail rabbit papillomavirus (CRPV) can protect against experimental CRPV infection. J Virol. 69:3959-63.

Bures R, Morris L, Williamson C, Ramjee G, Deers M, Fiscus SA, Abdool-Karim S, Montefiori D C. (2002) Regional clustering of shared neutralization determinants on primary isolates of clade C human immunodeficiency virus type 1 from South Africa. J Virol. 76:2233-44.

Casal J I, Langeveld J P, Cortes E, Schaaper W W, van Dijk E, Vela C, Kamstrup S, Meloen R H. (1995) Peptide vaccine against canine parvovirus: identification of two neutralization subsites in the N terminus of VP2 and optimization of the amino acid sequence. J Virol. 69:7274-7.

Chen X, Scala G, Quinto I, Liu W, Chun T W, Justement J S, Cohen O J, vanCott T C, Iwanicki M, Lewis M G, Greenhouse J, Barry T, Venzon D, Fauci A S. (2001) Protection of rhesus macaques against disease progression from pathogenic SHIV-89.6PD by vaccination with phage-displayed HIV-1 epitopes. Nat Med. 7:1225-31.

Christensen N D, Dillner J, Eklund C, Carter J J, Wipf G C, Reed C A, Cladel N M, Galloway D A. (1996) Surface conformational and linear epitopes on HPV-16 and HPV-18 L1 virus-like particles as defined by monoclonal antibodies. Virology. 223:174-84.

Da Silva D M, Pastrana D V, Schiller J T, Kast W M. (2001) Effect of preexisting neutralizing antibodies on the anti-tumor immune response induced by chimeric human papillomavirus virus-like particle vaccines. Virology. 290:350-60.

Dawson W O, Beck D L, Knorr D A, Grantham G (1986) cDNA cloning of the complete genome of tobacco mosaic virus and production of infectious transcripts. Proc. Natl. Acad. Sci. USA 83:1832-1836.

Day P M, Roden R B, Lowy D R, Schiller J T. (1998) The papillomavirus minor capsid protein, L2, induces localization of the major capsid protein, L1, and the viral transcription/replication protein, E2, to PML oncogenic domains. J Virol. 72:142-50.

D'Souza M P, Livnat D, Bradac J A, Bridges S H. (1997) Evaluation of monoclonal antibodies to human immunodeficiency virus type 1 primary isolates by neutralization assays: performance criteria for selecting candidate antibodies for clinical trials. AIDS Clinical Trials Group Antibody Selection Working Group. J Infect Dis. 175:1056-62.

Eckhart L, Raffelsberger W, Ferko B, Klima A, Purtscher M, Katinger H, Ruker F. (1996) Immunogenic presentation of a conserved gp41 epitope of human immunodeficiency virus type 1 on recombinant surface antigen of hepatitis B virus. J Gen Virol. 77:2001-8.

Einstein M H, Goldberg G L (2002) Human papillomavirus and cervical neoplasia. Cancer Investigation 20:1080-1085.

Engelbrecht S, de Villiers T, Sampson C C, zur Megede J, Barnett S W, van Rensburg E J. (2001) Genetic analysis of the complete gag and env genes of HIV type 1 subtype C primary isolates from South Africa. AIDS Res Hum Retroviruses. 17:1533-47.

Evans T G, Bonnez W, Rose R C, Koenig S, Demeter L, Suzich J A, O'Brien D, Campbell M, White W I, Balsley J, Reichman R C. (2001) A Phase 1 study of a recombinant viruslike particle vaccine against human papillomavirus type 11 in healthy adult volunteers. J Infect Dis. 183:1485-93.

Fehr T, Naim H Y, Bachmann M F, Ochsenbein A F, Spielhofer P, Bucher E, Hengartner H, Billeter M A, Zinkemagel R M. (1998) T-cell independent IgM and enduring protective IgG antibodies induced by chimeric measles viruses. Nat Med. 4:945-8.

Ferko B, Katinger D, Grassauer A, Egorov A, Romanova J, Niebler B, Katinger H, Muster T. (1998) Chimeric influenza virus replicating predominantly in the murine upper respiratory tract induces local immune responses against human immunodeficiency virus type 1 in the genital tract. J Infect Dis. 178:1359-68.

Fitchen J, Beachy R N, Hein M B. (1995) Plant virus expressing hybrid coat protein with added murine epitope elicits autoantibody response. Vaccine. 13:1051-7.

Gallo R C, Bumy A, Zagury D (2002) Targeting Tat and IFNα as a therapeutic AIDS vaccine. DNA Cell Biol. 21: 611-618.

Galloway D A (1996). Surface conformational and linear epitopes on HPV-16 and HPV-18 L1 virus-like particles as defined by monoclonal antibodies. Virology 223:174-184.

Gaukroger J M, Chandrachud L M, O'Neil B W, Grindlay G J, Knowles G, Campo M S. (1996) Vaccination of cattle with bovine papillomavirus type 4 L2 elicits the production of virus-neutralizing antibodies. J Gen Virol. 77:1577-83.

Ghim S, Christensen N D, Kreider J W, Jenson A B. (1991) Comparison of neutralization of BPV-1 infection of C127 cells and bovine fetal skin xenografts. Int J Cancer. 49:285-9.

Gibbs, A., (1999) Evolution and origins of tobamoviruses. Philosophical Transactions of the Royal Society of London Series B-Biological Sciences 354:593-602.

Gibbs, A. J., Tobamovirus Group. CMI/AAB Descriptions of Plant Viruses, 1977. 184: p. 1-6.

Giroglou T, Florin L, Schafer F, Streeck R E, Sapp M. (2001) Human papillomavirus infection requires cell surface heparan sulfate. J Virol. 75:1565-70.

Harro C D, Pang Y Y, Roden R B, Hildesheim A, Wang Z, Reynolds M J, Mast T C, Robinson R, Murphy B R, Karron R A, Dillner J, Schiller J T, Lowy D R. (2001) Safety and immunogenicity trial in adult volunteers of a human papillomavirus 16 L1 virus-like particle vaccine. J Natl Cancer Inst. 93:284-92.

He Y, Honnen W J, Krachmarov C P, Burkhart M, Kayman S C, Corvalan J, Pinter A. (2002) Efficient isolation of novel human monoclonal antibodies with neutralizing activity against HIV-1 from transgenic mice expressing human Ig loci. J Immunol. 169:595-605.

Isaacson M (2001) Viral hemorrhagic fever hazards for travelers in Africa. Clinical Infectious Diseases 33:1707-12.

Jaegle, M. and M. H. Van Regenmortel, (1985) Use of ELISA for measuring the extent of serological cross-reactivity between plant viruses. J Virol Methods11:189-98.

Jeang K T, Xiao H, Rich E A (1999) Multifaceted activities of the HIV transactivator of transcription Tat. J Biol. Chem. 274:28837-28840.

Kawana K, Kawana Y, Yoshikawa H, Taketani Y, Yoshiike K, Kanda T. (2001 a) Nasal immunization of mice with peptide having a cross-neutralization epitope on minor capsid protein L2 of human papillomavirus type 16 elicit systemic and mucosal antibodies. Vaccine. 19:1496-502.

Kawana Y, Kawana K, Yoshikawa H, Taketani Y, Yoshiike K, Kanda T. (2001b) Human papillomavirus type 16 minor capsid protein 12 N-terminal region containing a common neutralization epitope binds to the cell surface and enters the cytoplasm. J Virol. 75:2331-6.

Kawana K, Matsumoto K, Yoshikawa H, Taketani Y, Kawana T, Yoshiike K, Kanda T. (1998a) A surface immunodeterminant of human papillomavirus type 16 minor capsid protein L2. Virology. 245:353-9.

Kawana K, Yoshikawa H, Taketani Y, Yoshiike K, Kanda T. (1998b) In vitro construction of pseudovirions of human papillomavirus type 16: incorporation of plasmid DNA into reassembled L1/L2 capsids. J Virol. 72:10298-300.

Kawana K, Yoshikawa H, Taketani Y, Yoshiike K, Kanda T. (1999) Common neutralization epitope in minor capsid protein L2 of human papillomavirus types 16 and 6. J Virol. 73:6188-90.

Kawana K, Yasugi T, Kanda T, Kino N, Oda K, Okada S, Kawana Y, Nei T, Takada T, Toyoshima S, Tsuchiya A, Kondo K, Yoshikawa H, Tsutsumi O, Taketani Y (2003) Vaccine in press. Keegan K, Collett M S. (1986) Use of bacterial expression cloning to define the amino acid sequences of antigenic determinants on the G2 glycoprotein of Rift Valley fever virus. J Virol. 58:263-70.

Kirnbauer R, Taub J, Greenstone H, Roden R, Durst M, Gissmann L, Lowy D R, Schiller J T. (1993) Efficient self-assembly of human papillomavirus type 16 L1 and L1-L2 into virus-like particles. J Virol. 67:6929-36.

Kirnbauer R, Chandrachud L M, O'Neil B W, Wagner E R, Grindlay G J, Armstrong A, McGarvie G M, Schiller J T, Lowy D R, Campo M S. (1996) Virus-like particles of bovine papillomavirus type 4 in prophylactic and therapeutic immunization. Virology. 219:37-44.

Krebbers E, Bosch D, van der Kerckhove J (1992) Prospects and progress in the production of foreign proteins and peptides in plants. Plant Protein Engineering (PR Shewery and S Gutteridge, eds) Cambridge University Press, Cambridge, pp316-324.

Langeveld J P, Martinez-Torrecuadrada J, Boshuizen R S, Meloen R H, Ignacio Casal J. (2001) Characterisation of a protective linear B cell epitope against feline parvoviruses. Vaccine. 19:2352-60.

Letvin N L (2002) Strategies for an HIV vaccine. J Clin. Invest. 109:15-20.

Letvin N L, Barouch D H, Montefiori D C. (2002) Prospects for vaccine protection against HIV-1 infection and AIDS. Annu Rev Immunol. 20:73-99.

Letvin N L, Robinson S, Rohne D, Axthelm M K, Fanton J W, Bilska M, Palker T J, Liao H X, Haynes B F, Montefiori D C (2001) Vaccine-elicited V3 loop-specific antibodies in rhesus monkeys and control of a simian-human immunodeficiency virus expressing a primary patient human immunodeficiency virus type 1 isolate envelope. J Virol. 75:4165-75.

Lewis A D, Chen R, Montefiori D C, Johnson P R, Clark K R (2002). Generation of neutralizing activity against human immunodeficiency virus type 1 in serum by antibody transfer. J Virol. 76:8769-8775.

Liu W J, Gissmann L, Sun X Y, Kanjanahaluethai A, Muller M, Doorbar J, Zhou J. (1997) Sequence close to the N-terminus of L2 protein is displayed on the surface of bovine papillomavirus type 1 virions. Virology 227:474-83.

London S D, Schmaljohn A L, Dalrymple J M, Rice C M. (1992) Infectious enveloped RNA virus antigenic chimeras. Proc Natl Acad Sci USA. 89:207-11.

Marusic C, Rizza P, Lattanzi L, Mancini C, Spada M, Belardelli F, Benvenuto E, Capone I. (2001) Chimeric plant virus particles as immunogens for inducing murine and human immune responses against human immunodeficiency virus type 1. J Virol. 75:8434-9.

Mascola J R. (2002) Passive transfer studies to elucidate the role of antibody-mediated protection against HIV-1. Passive transfer studies to elucidate the role of antibody-mediated protection against HIV-1. Vaccine. 20:1922-5.

McMichael A, Mwau M, Hanke T. (2002) Design and tests of an HIV vaccine. Br Med Bull. 62:87-98.

Moulard M, Phogat S K, Shu Y, Labrijn A F, Xiao X, Binley J M, Zhang M Y, Sidorov I A, Broder C C, Robinson J, Parren P W, Burton D R, Dimitrov D S. (2002) Broadly cross-reactive HIV-1-neutralizing human monoclonal Fab selected for binding to gp120-CD4-CCR5 complexes. Proc Natl Acad Sci USA. 99:6913-8.

Pastrana D V, Vass W C, Lowy D R, Schiller J T. (2001) NHPV16 VLP vaccine induces human antibodies that neutralize divergent variants of HPV16. Virology 279:361-9. Pelcher L E, Halasa M C (1982) An RNA plant virus vector or portion thereof, a method of construction thereof, and a method of producting a gene-derived product therefrom. European Patent Application 067,533

Pelham H R B (1978) Leaky UAG stop codon in tobacco mosaic virus RNA. Nature 272:469-471.

Robinson H L (2002) New hope for an HIV vaccine. Nature Reviews Immunology 2:239-250.

Roden R B, Weissinger E M, Henderson D W, Booy F, Kirnbauer R, Mushinski J F, Lowy D R, Schiller J T (1994) Neutralization of bovine papillomavirus by antibodies to L1 and L2 capsid proteins. J Virol. 68:7570-4.

Roden R B, Greenstone H L, Kirnbauer R, Booy F P, Jessie J, Lowy D R, Schiller J T. (1996) In vitro generation and type-specific neutralization of a human papillomavirus type 16 virion pseudotype. J Virol. 70:5875-83.

Savelyeva N, Munday R, Spellerberg M B, Lomonossoff G P, Stevenson F K (2001) Plant viral genes in DNA idiotypic vaccines activate linked CD4+ T-cell mediated immunity against B-cell malignancies. Nat Biotechnol. 19:760-4.

Scala G, Chen X, Liu W, Telles J N, Cohen O J, Vaccarezza M, Igarashi T, Fauci AS. (1999) Selection of HIV-specific immunogenic epitopes by screening random peptide libraries with HIV-1-positive sera. J Immunol. 162:6155-61.

Schiller J T (1999) Papillomavirus-like particle vaccines for cervical cancer. Mol Med Today. 5:209-15.

Skuzeski J M, Nichols L M, Gesteland R F, Atkins J F (1991) The signal for a leaky UAF stop codon in several plant viruses includes that two downstream codons. J Mol. Biol. 365-373.

Stiegler G, Kunert R, Purtscher M, Wolbank S, Voglauer R, Steindl F, Katinger H. (2001) A potent cross-clade neutralizing human monoclonal antibody against a novel epitope on gp41 of human immunodeficiency virus type 1. AIDS Res Hum Retroviruses. 17:1757-65.

Stone K M, Karen K L, Sternberg M R, McQuilland G M, Poon A D, Unger E R, Reeves W C (2002) Seroprevalence of human papillomavirus type 16 infection in the United States. Journal of Infectious Diseases 186:1396-1402.

Storni T, Lechner F, Erdmann I, Bachi T, Jegerlehner A, Dumrese T, Kundig T M, Ruedl C, Bachmann M F. (2002) Critical role for activation of antigen-presenting cells in priming of cytotoxic T cell responses after vaccination with virus-like particles. J Immunol. 168:2880-6.

Stubbs, G., (1999) Tobacco mosaic virus particle structure and the initiation of disassembly. Philos Trans R Soc Lond B Biol Sci, 354:551-7.

Suzich J A, Ghim S J, Palmer-Hill F J, White W I, Tamura J K, Bell J A, Newsome J A, Jenson A B, Schlegel R. (1995) Systemic immunization with papillomavirus L1 protein completely prevents the development of viral mucosal papillomas. Proc Natl Acad Sci U S A. 92:11553-7.

Thompson G H, Roman A. (1987) Expression of human papillomavirus type 6 E1, E2, L1 and L2 open reading frames in Escherichia coli. Gene. 56:289-95.

Tikhonov I, Ruckwardt T J, Hatfield G S, Pauza C D (2003) Tat-neutralizing antibodies in vaccinated Macaques. J Virol. 77:3157-3166.

Unckell F, Streeck R E, Sapp M. (1997) Generation and neutralization of pseudovirions of human papillomavirus type 33. J Virol. 71:2934-9.

Van Regenmortel, M. H. V. (1999) The antigenicity of tobacco mosaic virus. Philos Trans R Soc Lond B Biol Sci, 354:559-68.

Wilson J A, Hevey M, Bakken R, Guest S, Bray M, Schmaljohn A L, Hart M K. (2000) Epitopes involved in antibody-mediated protection from Ebola virus. Science. 287:1664-6.

Xu W, Hofmann-Lehmann R, McClure H M, Ruprecht R M. (2002) Passive immunization with human neutralizing monoclonal antibodies: correlates of protective immunity against HIV. Vaccine. 20:1956-60.

Yuan H, Estes P A, Chen Y, Newsome J, Olcese V A, Garcea R L, Schlegel R. (2001) Immunization with a pentameric L1 fusion protein protects against papillomavirus infection. J Virol. 75:7848-53.

zur Hausen H (1999) Papillomaviruses in human cancers. Proc Assoc Am Physicians. 111:581-7.

Zwick M B, Labrijn A F, Wang M, Spenlehauer C, Saphire E O, Binley J M, Moore J P, Stiegler G, Katinger H, Burton D R, Parren P W. (2001) Broadly neutralizing antibodies targeted to the membrane-proximal external region of human immunodeficiency virus type 1 glycoprotein gp41. J Virol. 75:10892-905.

Zwick M B, Wang M, Poignard P, Stiegler G, Katinger H, Burton D R, Parren P W. (2001) Neutralization synergy of human immunodeficiency virus type 1 primary isolates by cocktails of broadly neutralizing antibodies. J Virol. 75:12198-208.

### References for HPV Epitope Peptides

HPV ep1

Kawana K, Yoshikawa H, Taketani Y, et al. Common neutralization epitope in minor capsid protein L2 of human papillomavirus types 16 and 6 J VIROL 73 (7): 6188-6190 July 1999

HPV ep2

Konya J, Eklund C, afGeijersstam V, et al. Identification of a cytotoxic T-lymphocyte epitope in the human papillomavirus type 16 E2 protein J GEN VIROL 78: 2615-2620 Part 10 October 1997

HPV ep3

Bartholomew J S, Stacey S N, Coles B, et al. Identification Of A Naturally Processed HLA A0201—Restricted Viral Peptide From Cells Expressing Human Papillomavirus Type-16 E6 Oncoprotein Eur J Immunol 24 (12): 3175-3179 December 1994

HPV ep4

Villada I B, Beneton N, Bony C, et al. Identification in humans of HPV-16 E6 and E7 protein epitopes recognized by cytolytic T lymphocytes in association with HLA-B18 and determination of the HLA-B18-specific binding motif EUR J IMMUNOL 30 (8): 2281-2289 August 2000

HPV ep5

Azoury-Ziadeh R, Herd K, Fernando G J P, et al.T-Helper epitopes identified within the E6 transforming protein of cervical cancer-associated human papillomavirus type 16 VIRAL IMMUNOL 12 (4): 297-312 1999

HPV ep6

As for HPV ep5

HPV ep7

Tindle R W, Fernando G J P, Sterling J C, et al. A Public T-Helper Epitope Of The E7 Transforming Protein Of Human Papillomavirus-16 Provides Cognate Help For Several E7 B-Cell Epitopes From Cervical Cancer-Associated Human Papillomavirus Genotypes P Natl Acad Sci USA 88 (13): 5887-5891 July 1991

HPV ep8

Ressing M E, Sette A, Brandt R M P, et al. Human CTL Epitopes Encoded By Human Papillomavirus Type-16 E6 And E7 Identified Through In-Vivo And In-Vitro Immunogenicity Studies Of HLA-A-Asterisk-0201-Binding Peptides J Immunol 154 (11): 5934-5943 June 1 1995

HPV ep9

As for HPV ep8

HPV ep10

As for HPV ep8

HPV ep11

As for HPV ep4

HPV ep12

Garcia A M, Ortiz-Navarrete V F, Mora-Garcia M D, et al. Identification of peptides presented by HLA class I molecules on cervical cancer cells with HPV-18 infection IMMUNOL LETT 67 (3): 167-177 Apr. 15 1999

HPV ep13

As for HPV ep1

HPV ep14

Rudolf M P, Man S, Melief C J M, et al. Human T-cell responses to HLA-A-restricted high binding affinity peptides of human papillomavirus type 18 proteins E6 and E7 CLIN CANCER RES 7 (3): 788S-795S Suppl. S March 2001

HPV ep15

As for HPV ep14

HPV ep16

As for HPV ep14

HPV ep17

AS for HPV ep14

HPV ep18

Hohn H, Pilch H, Gunzel S, et al. Human papillomavirus type 33 E7 peptides presented by HLA-DR*0402 to tumor-infiltrating T cells in cervical cancer J VIROL 74 (14): 6632-6636 July 2000

What is claimed is:

1. An immunological reagent comprising a plant viral protein covalently bound to an epitope peptide having the same linear sequence as an immunologically recognized epitope of a human papilloma virus, human immunodeficiency virus, ebola virus, rift valley fever virus or parvovirus.

2. An immunological reagent of claim 1 wherein the epitope peptide contains a sequence selected from the group consisting of the peptide sequences of Table 1, the peptide sequences of Table 6, the peptide sequences of Table 7, the peptide sequences of Table 8,
HNTPVYKLDISEATQVE, ATQVEQHHRRTDNDSTA, GKLGLITNTIAGVAGLI,
VQPDGGQPAVRNERAT, MSDGAVQPDGGQPAVRNERA,
MSDGAVQPDGGQPAVRNERAT and KGTMDSGQTKREL.

3. A vaccine comprising the composition of claims 2, and a pharmaceutically acceptable carrier or excipient.

4. A method for eliciting an immune response in an animal comprising administering the vaccine of claim 3 to the animal.

5. A virus-like particle comprising a plurality of assembled protein subunits wherein each protein subunit is a plant viral coat protein covalently bound to an epitope peptide having the same linear sequence as an immunologically recognized epitope of a human papilloma virus, human immunodeficiency virus, ebola virus, rift valley fever virus or parvovirus.

6. A virus-like particle of claim 5 wherein said sequence selected from the group consisting of the peptide sequences of Table 1, the peptide sequences of Table 6, the peptide sequences of Table 7, the peptide sequences of Table 8, HNTPVYKLDISEATQVE, ATQVEQHHRRTDNDSTA, GKLGLITNTIAGVAGLI, VQPDGGQPAVRNERAT, MSDGAVQPDGGQPAVRNERA, MSDGAVQPDGGQPAVRNERAT and KGTMDSGQTKREL.

7. A vaccine comprising the composition of claim 5, and a pharmaceutically acceptable carrier or excipient.

8. A method for eliciting an immune response in an animal comprising administering the vaccine of claim 7 to the animal.

9. A plant virus comprising at least one plant viral coat protein covalently bound to an epitope peptide having the same linear sequence as an immunologically recognized epitope of a human papilloma virus, human immunodeficiency virus, ebola virus, rift valley fever virus or parvovirus.

10. A plant virus of claim 9 wherein said sequence is selected from the group consisting of the peptide sequences of Table 1, the peptide sequences of Table 6, the peptide sequences of Table 7, the peptide sequences of Table 8, HNTPVYKLDISEATQVE, ATQVEQHHRRTDNDSTA, GKLGLITNTIAGVAGLI, VQPDGGQPAVRNERAT, MSDGAVQPDGGQPAVRNERA, MSDGAVQPDGGQPAVRNERAT and KGTMDSGQTKREL.

11. A vaccine comprising the composition of claim 10 and a pharmaceutically acceptable carrier or excipient.

12. A method for eliciting an immune response in an animal comprising administering the vaccine of claim 11 to the animal.

13. The composition of claims 6 or 10 containing a plurality of different epitope peptides, each on a separate plant viral coat protein molecule.

14. A method for preparing an antibody against a papilloma virus, ebola virus, HIV virus, Rift Valley Fever virus or a parvovirus comprising;
exposing an animal to the vaccine of claim 3, 7 or 11,
recovering cells or body fluids from the animal, and
preparing an antibody from said cells or body fluids.

15. The method of 14 wherein the antibody is neutralizing.

16. A method for detecting a papilloma virus, ebola virus, HIV virus, Rift Valley Fever virus or a parvovirus comprising contacting an antibody produced by the method of claim 14 with a sample suspecting of containing a virus, and detecting the presence or absence of antibody binding to the virus.

17. A method for inducing an immune response in an animal against a peptide epitope comprising
coupling the peptide epitope to a first carrier antigen to make a first vaccine composition,
coupling the peptide epitope to a second carrier antigen, which is different from the first carrier antigen, to make a second vaccine composition,
immunizing the animal with the first vaccine composition,
at a later time, immunizing the animal with the second vaccine composition,
wherein the immune response to the peptide epitope is boosted greater than the boosting of either carrier antigen.

18. The method according to claim 17 further comprising;
coupling a second peptide epitope to a third carrier antigen to make a third vaccine composition,
coupling the second peptide epitope to a fourth carrier antigen, which is different from the third carrier antigen but may be the same as either the first carrier antigen or the second carrier antigen, to make a fourth vaccine composition,
immunizing an individual animal with the first vaccine composition and the third composition,
at a later time, immunizing the same individual animal with the second vaccine composition and the fourth composition.
wherein the immune responses to the first and second peptide epitope are boosted greater than the boosting of the carrier antigens.
